(19) United States
(12) Patent Application Publication
(10) Pub. No.: US 2019/0105369 A1
(43) Pub. Date: Apr. 11, 2019

(54) CD31SHED AGONISTS FOR USE IN THE PREVENTION AND/OR TREATMENT OF REPERFUSION INJURY

(71) Applicants: INSTITUT NATIONAL DE LA SANTE ET DE LA RECHERCHE MEDICAL (INSERM), PARIS (FR); UNIVERSITÉ PARIS DIDEROT - PARIS 7, PARIS (FR); UNIVERSITE PARIS 13 - PARIS NORD, VILLETANEUSE (FR)

(72) Inventors: Giuseppina CALIGIURI, PARIS (FR); Jean-Baptiste MICHEL, PARIS (FR); Antonino NICOLETTI, PARIS (FR)

(73) Assignees: INSTITUT NATIONAL DE LA SANTE ET DE LA RECHERCHE MEDICAL (INSERM), Paris (FR); UNIVERSITÉ PARIS DIDEROT - PARIS 7, Paris (FR); UNIVERSITE PARIS 13 - PARIS NORD, Villetaneuse (FR)

(21) Appl. No.: 16/087,223

(22) PCT Filed: Mar. 21, 2017
(86) PCT No.: PCT/EP2017/056733

§ 371 Date: Sep. 21, 2018

(30) Foreign Application Priority Data
Mar. 21, 2016 (EP) 16305311.9

(51) Int. Cl. A61K 38/17; A61P 9/10

(52) U.S. Cl. CPC A61K 38/1709; A61P 9/10

(57) ABSTRACT

Disclosed are CD31^{shed} agonists for use in the prevention and/or treatment of reperfusion injury. These CD31^{shed} agonists are peptides or peptidomimetics thereof that are able to restore CD31 signaling in cells bearing a truncated form of CD31 called CD31^{shed}. The CD31^{shed} agonists particularly protect from organ damages caused by reperfusion used to treat ischemia.

### FIELD OF THE INVENTION

The present invention relates to the prevention and/or treatment of reperfusion injury.

### BACKGROUND

Ischemia is a condition in which blood supply to tissues is severely restricted or arrested, causing a shortage of oxygen and glucose needed for cellular metabolism. Ischemia may for example be due to the presence of a critical stenosis of an artery, to the formation of an occlusive thrombus on an atherosclerotic plaque or to the clamping of a vessel. Artery thrombosis is also characterized by blood stasis in the vessels, downstream the occlusion, including both arteries and veins. Ischemia is the underlying process of life-threatening clinical conditions such as myocardial infarction, stroke, mesenteric ischemia or ischemia of the lower limbs. In highly aerobic tissues, such as heart, brain and intestine, irreversible ischemic damage to tissues can occur very shortly.

During ischemia, mitochondria are gradually no longer able to produce ATP via the citric acid cycle. They switch to an anaerobic glycolysis, which results in lactate production and the acidification of the intracellular medium leading to a major tissue metabolic impairment. In the heart muscle, these phenomena result in the inhibition of mechanical contraction, leading to an acute heart muscle failure. In mesenteric ischemia, they are often associated with a lesion of the intestinal epithelium and loss of mucus protection, leading to a bacterial translocation in blood.

Prolonged ischemia results in necrosis of the area corresponding to the low-perfused or non-perfused tissue and may irreversibly compromise the function of the organ.

The only strategy able to stop the tissue damage caused by ischemia is reperfusion. Reperfusion may be mechanical (for example angioplasty or bypass surgery), pharmacological (perfusion with a fibrinolytic agent) or natural (endogenous fibrinolysis). However, although reperfusion of an ischemic tissue is essential to its survival, the restoration of blood supply in an ischemic tissue causes additional damage, called reperfusion injury. Additional damage caused by reperfusion in an ischemic organ is undoubtedly related to the acute oxygen supply in an environment wherein the cells had changed their metabolism to adapt themselves to the absence of oxygen.

Inhibitors of adhesion molecules (for example RhuMab CD18 antibody, Hu23F2G antibody and glycoprotein rPSLG-Ig) have been proposed as candidate drug. Disappointingly, all the clinical trials based on the use of such molecules, in conjunction with revascularization, failed to limit the size of myocardial infarction.

CD31 consists of a single chain 130-kDa glycoprotein comprising six Ig-like extracellular domains, a short transmembrane segment and a cytoplasmic tail. The cytoplasmic tail contains two important tyrosine-based motifs (around Y663 and Y686) that act as Immunoreceptor Tyrosine-based Inhibitory Motifs (ITIMs).

The intracellular CD31 ITIMs are not phosphorylated in resting conditions because CD31 does not possess an intrinsic kinase activity. CD31 molecules bind to each other via a trans-homophilic liaison of the Ig-like domains 1-2 between interacting cells. This trans-homophilic binding is required to trigger the clustering of the CD31 molecules on the membrane plane, which in turn requires a cis-homophilic juxta-membrane sequence. The phosphorylation of the CD31 intracellular ITIMs becomes then possible because its ITIMs can be exposed to the activity of the tyrosine kinases that are carried close by other cluster-associated membrane receptors (such for instance the T cell receptor). The phosphorylation of the intracellular ITIMs triggers the recruitment and activation of intracellular SH2-containing phosphatases. Depending on the signaling adaptors associated to the closest membrane receptor, the activation of SH2-containing phosphatases can lead to either the activation of signaling cascades (e.g. GAB/ERK/MAPK, driving adherence, survival and growth of endothelial cells, foxp3 expression and differentiation of lymphocytes into the regulatory phenotype, driving active cell-cell detachment) or their inhibition (e.g. JAK/STAT, preventing leukocyte and platelet activation). Accordingly, the function of CD31 varies upon the cell type.

WO2010/000741 discloses that the assumed loss of CD31 on activated/memory T lymphocytes is actually incomplete and results from shedding of CD31 between the 5th and the 6th extracellular Ig-like domains. The shed extracellular domain of CD31 (referred to as “soluble CD31”) is then released into the circulation, where it is present together with a soluble splice variant of CD31. The remaining small CD31 ectodomain which remains anchored to the membrane after shedding is referred to as “CD31^{shed}”. This document also discloses peptides corresponding to juxta-membrane amino acids of the ectodomain of CD31 that are able to rescue the physiological immunoregulatory function of CD31, by bridging CD31^{shed}, via a strong homo-oligomerization. Such peptides are useful for the treatment of thrombotic or autoimmune disorders. As regard to thrombotic disorders, these peptides indeed directly act on atherothrombosis and atherosclerosis, for example by preventing acceleration of plaque growth, arterial dissection, and formation of aneurysm.

WO2013/190014 further discloses specific peptides of 8 amino acids, within the membrane juxta-proximal part of extracellular CD31, which hold useful for the treatment of thrombotic or inflammatory disorders and display physic-chemical properties that are more suitable for drug development. Peptide P8RI for example reduces human platelet aggregation and thrombin generation, thereby preventing thrombotic occlusion.

Thrombotic disorders, such as atherothrombosis or atherosclerosis, which are not treated, may lead to an ischemia and thus precede ischemia. Stricto senso, ischemia is indeed not a thrombotic disorder and indicates the interruption of blood flow into an organ, not the cause of it.

There is therefore still a need to provide solutions for the prevention and/or treatment of reperfusion injury, in particular for at least reducing organ damage caused by reperfusion.

### DESCRIPTION OF THE INVENTION

Surprisingly, the Inventors have found that a CD31^{shed} agonist able to bind to the truncated form of CD31 present on a cell surface (herein called CD31^{shed}), thereby triggering the CD31 signaling, is useful in acute pathological situations involving the explosive activation of neutrophils, such as reperfusion injury. Said CD31^{shed} agonist is preferably a synthetic peptide, such as the peptide P8RI of sequence SEQ ID NO: 6 consisting of D-enantiomer amino acids.

As a matter of fact, CD31 is highly expressed on the surface of granulocytes. Moreover, the inventors have found that, in granulocytes and endothelial cells, CD31 is also cleaved upon activation of the cells. The cleavage of CD31 abolishes its homophilic CD31-CD31 homeostatic functions.

Since granulocytes are the main cells involved in the inflammatory tissue damage carried by reperfusion injury, this might explain the therapeutic effect of a CD31^{shed} agonist in the prevention and/or treatment of reperfusion injury. Nevertheless, taking into account the complexity of the cellular events involved in reperfusion, the therapeutic effect of the solely administration of a CD31^{shed} agonist that mainly targets the inflammatory processes mediated by the granulocytes (and not the associated metabolic processes) is highly surprising.

It is believed that the CD31^{shed} agonist advantageously does not deplete or definitively suppress the targeted blood elements (essentially neutrophils and platelets in the setting of acute reperfusion) and allows preserving their physiologic function in wound healing.

The inventors have indeed shown that the solely administration of a CD31^{shed} agonist to an animal suffering from ischemia, prior to reperfusion, enables to significantly decrease the extent of necrotic damage. As a matter of fact, the loss of CD31 (in CD31 knock-out mice) increases tissue necrosis caused by reperfusion and mortality, in a mouse model of myocardial infarction (left anterior coronary ligation) (see for example FIG. 1). On the contrary, the administration of peptide P8RI during the ischemia-reperfusion period enables to strongly decrease the size of the necrotic areas by comparison to a non-treated control mouse.

Besides, in a mesenteric artery transient occlusion model, the administration of peptide P8RI not only prevented intestinal necrosis, but also the bacterial translocation, as shown by the arrest of the progressive increase in the level of free DNA, the decrease in the concentration of intra-digestive hemoglobin and in the LPS secretion in blood and the histological conservation of mucus on the intestinal villosities.

The present invention thus relates to a CD31^{shed} agonist for use in the prevention and/or treatment of reperfusion injury, wherein said CD31^{shed} agonist binds to CD31^{shed} present on a cell surface, said binding leading to the phosphorylation of at least one CD31^{shed} ITIM tyrosine.

The use of a CD31^{shed} agonist according to the invention particularly enables to:
stop or slow down the progression of necrosis, for example to decrease the size of the necrotic areas, in particular in the ischemic tissue or organ, during warm reperfusion (for example in heart, brain, intestine, kidneys and/or limbs),
increase the chances of organ survival after the reperfusion,
stop or limit the vascular lesion(s) and/or intra-tissue hemorrhage of the reperfused tissue (for example, stop or limit hemorrhagic transformation of an ischemic stroke in brain; stop or limit hemorrhagic necrosis of the epithelium, in particular in the intestine) and/or
limit bacterial translocation (in particular in the intestinal epithelium, during reperfusion further to a mesenteric ischemia), and/or
increase the cold ischemia time and/or improve the recovery of a graft, in the case of a tissue or organ transplantation.

The present invention particularly relates to the CD31^{shed} agonist for use in the prevention and/or treatment of reperfusion injury as defined above, wherein reperfusion is used for treating ischemia, such as myocardial infarction, ischemic colitis, mesenteric ischemia, stroke, ischemia of the lower limbs, visceral ischemia resulting from acute hypovolemia, ischemia resulting from inflammatory conditions, ischemia inherent to a medical procedure (such as ischemia inherent to an extracorporeal circulation blood excluding part of the arterial branches, ischemia inherent to a visceral and/or aortic surgery, cold ischemia, and/or ischemia inherent to warm reperfusion of a graft directed to organ transplantation) and their combinations.

The CD31^{shed} agonist is preferably used or administered:
before and/or during the reperfusion, preferably before and/or during the ischemia-reperfusion period, when possible,
intravenously, including under continuous infusion,
continuously during reperfusion, in particular when ischemia could not be covered, and/or
during a short period of time, for example for at most 48 hours, more preferably at most 24 h.

The present invention also relates the a CD31^{shed} agonist for use as defined above, wherein a first dose of said CD31^{shed} agonist is used as a single dose, preferably before ischemia (for example when ischemia is inherent to a medical procedure such as aortic and/or visceral surgery, cold ischemia and/or warm reperfusion of a graft directed to organ transplantation) and a second dose of said CD31^{shed} agonist is used continuously, in particular during the time of ischemia-reperfusion.

The CD31^{shed} agonist for use as defined above is preferably:
a) a peptide selected in the group consisting of:
   (i) a peptide consisting of a fragment of 3 to 15 amino acids of the sequence defined by amino acids 579 to 601 of sequence SEQ ID NO: 1,
   (ii) a peptide consisting of a fragment of 3 to 15 amino acids of a sequence corresponding to the amino acids 579 to 601 of sequence SEQ ID NO: 1 in a non-human mammalian CD31,
   (iii) a peptide of 3 to 15 amino acids consisting of a sequence at least 70% identical to the sequence of peptide (i),
   (iv) a peptide consisting of a retro-inverso sequence of peptide (i), (ii) or (iii), and
   (v) the peptide (i), (ii), (iii) or (iv) comprising at least one or at least one further chemical modification,

or
b) a peptidomimetic of said peptide a).

Preferably, said peptide is soluble in water and/or resistant to peptidase.

In one embodiment, the peptide for use as defined above comprises at least one chemical modification to improve its stability and/or bioavailability.

In a preferred embodiment, said peptide comprises at least one artificial amino acid, said artificial amino acid being preferably selected from the group consisting of a D-enantiomer amino acid, beta-methyl amino acid, alpha-substituted alpha-amino acid and amino acid analog.

In a still preferred embodiment, said peptide (v) comprises at least one amino acid in the D-enantiomer form.

Examples of peptide for use as defined above are a peptide of sequence SEQ ID NO: 2, a peptide of sequence SEQ ID NO: 3 (also called PepReg CD31), a peptide of sequence SEQ ID NO: 4, a peptide of sequence SEQ ID NO: 5 (also called P8F), a peptide of sequence SEQ ID NO: 6 consisting of D-enantiomer amino acids (also called P8RI), a peptide of sequence SEQ ID NO: 7 and a peptide of sequence SEQ ID NO: 8 consisting of D-enantiomer amino acids.

A preferred peptide for use as defined above is a peptide of sequence SEQ ID NO: 5, or a peptide of sequence SEQ ID NO: 6 consisting of D-enantiomer amino acids.

CD31^{shed} Agonist

By “CD31^{shed} agonist”, it is herein meant a compound that binds to CD31^{shed} present on a cell surface, said binding leading to the phosphorylation of at least one CD31^{shed} ITIM tyrosine.

An ITIM (Immunoreceptor Tyrosine-based Inhibitory Motif) consists of the consensus sequence T/L/I/V/S-x-V-x-x-L/V/I, wherein x represents any amino acid.

CD31 (as well as CD31^{shed}) generally comprise two ITIMs.

For example, the two human CD31 ITIMs are 663 ITIM of sequence VQYTEV and 686 ITIM of sequence TVYSEV.

In sequence SEQ ID NO: 1, the tyrosine of 663 ITIM is in position 690 and those of 686 ITIM is in position 713.

For example, the two murine CD31 ITIMs are 663 ITM of sequence VEYTEV and 686 ITIM of sequence TVYSEI.

The binding of the CD31^{shed} agonist to CD31^{shed} present on a cell surface leads preferably to the phosphorylation of at least the tyrosine of the 686 ITIM of CD31^{shed}.

The binding of a compound to CD31^{shed} present on a cell surface may be assessed by any method well-known by the skilled person.

For example, the binding may be measured by plasmon surface resonance, flow cytometry or beta-imager.

In a preferred embodiment, the binding of a compound to CD31^{shed} present on a cell surface is assessed as follows: the compound to be tested, or an irrelevant analogue as negative control, is bound to a fluorescent probe (e.g. fluoresceine). The compound is incubated at consecutive dilutions (for example 1, 10, 100 μmol) with CD31+ cells (for example from a cell line, such as jurkat T cells, or primary cells, such as peripheral blood T cells) at a density of 10⁶ cells/ml in a saline buffer comprising Ca++ and Mg++ (HBSS, culture medium). Parallel conditions are incubated in the presence of a cell activator (e.g. TCR crosslinking, such as 1 μg/ml anti-CD3e antibodies+20 μg/ml secondary F(ab′)2 fragment if the cells are T lymphocytes). The reaction is stopped after 5 or 20 minutes by repeated washing steps with cold buffer. Cells are fixed with paraformaldehyde and washed again. Binding of the compound to be tested on individual cells is detected by the relative fluorescent signal using a flow cytometer. A greater signal for the specific compound as compared to the control and in the conditions comprising the cell activator as compared to resting cells indicates appropriate binding to CD31^{shed}.

The phosphorylation of at least one CD31^{shed} ITIM tyrosine may be assessed directly or indirectly, for example by measuring the phosphorylation of the intracellular SH-2 tyrosine phosphatase, which is phosphorylated by the agonist.

The phosphorylation of at least one CD31^{shed} ITIM tyrosine and/or of the intracellular SH-2 tyrosine phosphatase may be assessed by any method well-known by the skilled person.

For example, the phosphorylation of at least one CD31^{shed} ITIM tyrosine may be assessed as follows: the compound to be tested is bound to a fluorescent probe (e.g. fluoresceine). The compound is incubated at consecutive dilutions (1, 10, 100 μμmol) with CD31+ cells (for example cell lines, such as jurkat T cells, or primary cells, such as peripheral blood T cells) at a density of 10⁶ cells/ml in a saline buffer comprising Ca++ and Mg++ (HBSS, culture medium). Parallel conditions are incubated in the presence of a cell activator (e.g. TCR crosslinking, such as 1 μg/ml anti CD3e antibodies +20 μg/ml secondary F(ab′)2 fragment if the cells are T lymphocytes). The reaction is stopped after 5 or 20 minutes by repeated washing steps with could buffer. Cells are fixed with paraformaldehyde, permeabilized with methanol containing buffer (e.g. PermBuffer III from BD biosciences) and washed again. Intracellular staining of CD31^{shed} ITIM tyrosine CD31 (for example pY686) or of the intracellular SH-2 tyrosine phosphatase (pSHP2 Y542) is performed using the appropriate fluorescent antibody, diluted in the permeabilization buffer during 30 minutes at 4° C. and in the dark. Cells are then repeatedly washed and the relative fluorescent signal is acquired on a flow cytometer.

The CD31^{shed} agonist preferably exerts in vitro a dose-dependent increase of the phosphorylation of at least one CD31^{shed} ITIM tyrosine and/or of intracellular SH-2 tyrosine phosphatase.

The phosphorylation of at least one CD31^{shed} ITIM tyrosine may also be assessed indirectly, by measuring the inhibition of neutrophil, platelet and/or endothelial cell activation.

The inhibition of neutrophil, platelet and/or endothelial cell activation may be assessed by any method well-known by the skilled person, for example wherein the CD31 agonist is added at the same time as the stimulus in parallel conditions.

For example, neutrophil activation can be evaluated by the increase in surface integrin expression. Percoll® purified peripheral blood neutrophils are primed with recombinant human IL-8 or TNFα and then fully activated with fMLP. After 30 minutes, the cells are stained with monoclonal antibodies directed surface markers, such as the beta 2 integrin CD11b, which dramatically increase upon cell activation, and analyzed by flow cytometry in the appropriate fluorescent channels.

Platelet activation can be evaluated by the release of CD62P (P-selectin). For example, platelet rich plasma deriving from peripheral venous blood is stimulated with monoclonal antibodies directed against CD32A (activating Fc receptor). The reaction is stopped after 30 minutes, by the addition of EDTA. Test tubes are then centrifuged and the supernatant is used for soluble CD62P measure (by commercially available ELISA or bead-based tests).

Endothelial cell activation can be evaluated by the expression of VCAM-1. For example, human primary endothelial cells (from the umbilical vein or coronary artery, both being commercially available from Promocell), passage 2-3, are cultured onto sterile coverlips and stimulated overnight with TNFα. The next days the coverslip is fixed and stained with monoclonal antibodies directed against VCAM-1 (CD106) and appropriate fluorescently labelled secondary antibody prior to nuclear counterstaining and analysis of VCAM-1 positive cells by fluorescence microscopy.

The CD31^{shed} agonist preferably exerts in vitro a dose-dependent inhibition of neutrophil, platelet and/or endothelial cell activation.

In a preferred embodiment, the CD31^{shed} agonist is a peptide or a peptidomimetic thereof.

### Peptide Used as CD31^{shed} Agonist

In one embodiment of the invention, the CD31^{shed} agonist is a peptide.

The peptide is preferably a peptide as disclosed in WO2010/000741 or WO2013/190014.

Preferably, the CD31 peptide is a synthetic peptide.

By a “synthetic peptide”, it is intended that the peptide is not present within a living organism, e.g. within human body.

The synthetic peptide may be part of a composition or a kit.

The synthetic peptide is preferably purified.

The peptide may be selected in the group consisting of:

(i) a peptide consisting of a fragment of 3 to 15 amino acids of the sequence defined by amino acids 579 to 601 of sequence SEQ ID NO: 1,

(ii) a peptide consisting of a fragment of 3 to 15 amino acids of a sequence corresponding to the amino acids 579 to 601 of sequence SEQ ID NO: 1 in a non-human mammalian CD31,

(iii) a peptide of 3 to 15 amino acids consisting of a sequence at least 70% identical to the sequence of peptide (i),

(iv) a peptide consisting of a retro-inverso sequence of peptide (i), (ii) or (iii), and

(v) the peptide (i), (ii), (iii) or (iv) comprising at least one or at least one further chemical modification, preferably at least one amino acid in the D-enantiomer form.

A “fragment” refers herein to a sequence of consecutive amino acids. For example, a fragment may be a fragment of 3, 4, 5, 6, 7, 8, 9, 10, 11, 12, 13, 14 or 15 amino acids.

The sequence defined by amino acids 579 to 601 of sequence SEQ ID NO: 1 is sequence SEQ ID NO: 12.

Thus, the peptide may consist of a fragment of 3 to 15 amino acids of sequence SEQ ID NO: 12.

The peptide may also consist of a fragment of 3 to 15 amino acids of a sequence corresponding to sequence SEQ ID NO: 12 in a non-human mammalian CD31.

Non-limiting examples of non-human mammalian CD31 are the murine CD31 of sequence SEQ ID NO: 9, the bovine CD31 of sequence SEQ ID NO: 10 and the pig CD31 of sequence SEQ ID NO: 11.

The person skilled in the art can easily identify a sequence corresponding to the amino acids 579 to 601 of sequence SEQ ID NO: 1, i.e. to sequence SEQ ID NO: 12, in a non-human mammalian CD31 protein, for example by performing a sequence alignment between sequence SEQ ID NO: 1 and the sequence of said non-human mammalian CD31 protein, for example with one of sequences SEQ ID NO: 9, SEQ ID NO: 10 and SEQ ID NO: 11.

Methods for sequence alignment and determination of sequence identity are well known in the art, for example using publicly available computer software such as BioPerl, BLAST, BLAST-2, CS-BLAST, FASTA, ALIGN, ALIGN-2, LALIGN, Jaligner, matcher or Megalign (DNASTAR) software and alignment algorithms such as the Needleman-Wunsch and Smith-Waterman algorithms.

The sequence of the CD31 peptide according to the invention is preferably derived from the sequence of human CD31 or murine CD31.

The peptide may also be a peptide of 3 to 15 amino acids consisting of a sequence at least 70%, at least 75%, at least 80%, at least 85% or at least 90% identical to the sequence of peptide (i), i.e. to the sequence of a fragment of 3 to 15 amino acids of the sequence defined by amino acids 579 to 601 of sequence SEQ ID NO: 1.

A peptide sequence at least 70% identical to a given sequence of 4 to 6 amino acids differs from said given sequence of at most one amino acid.

A peptide sequence at least 70% identical to a given sequence of 7 to 9 amino acids differs from said given sequence of at most two amino acids.

A peptide sequence at least 70% identical to a given sequence of 10 to 13 amino acids differs from said given sequence of at most three amino acids.

A peptide sequence at least 70% identical to a given sequence of 14 or 15 amino acids differs from said given sequence of at most four amino acids.

By “a sequence at least x % identical to a reference sequence”, it is intended that the amino acid sequence of the subject peptide is identical to the reference sequence or differ from the reference sequence by up to 100-x amino acid alterations per each 100 amino acids of the reference sequence. In other words, to obtain a polypeptide having an amino acid sequence at least x % identical to a reference amino acid sequence, up to 100-x % of the amino acid residues in the subject sequence may be inserted, deleted or substituted with another amino acid.

Methods for comparing the identity of two or more sequences are well known in the art. For instance, programs available in the Wisconsin Sequence Analysis Package, version 9.1, for example the programs BESTFIT and GAP, may be used to determine the % identity between two polypeptide sequences. BESTFIT uses the “local homology” algorithm of Smith and Waterman and finds the best single region of similarity between two sequences. Other programs for determining identity between sequences are also known in the art, for instance the Needle program, which is based on the Needleman and

Wunsch algorithm, described in Needleman and Wunsch (1970) J. Mol Biol. 48:443-453, with for example the following parameters for polypeptide sequence comparison: comparison matrix: BLOSUM62, gap open penalty: 10 and gap extend penalty: 0.5, end gap penalty: false, end gap open penalty=10, end gap extend penalty=0.5; and the following parameters for polynucleotide sequence comparison: comparison matrix: DNAFULL; gap open penalty=10, gap extend penalty=0.5, end gap penalty: false, end gap open penalty=10, end gap extend penalty=0.5.

Peptides consisting of an amino acid sequence “at least 70%, 75%, 80%, 85%, or 90% identical” to a reference sequence may comprise mutations, such as deletions, insertions and/or substitutions compared to the reference sequence.

In case of substitutions, the substitution preferably corresponds to a conservative substitution as indicated in the Table 1 below. In a preferred embodiment, the peptide consisting of an amino acid sequence at least 70%, 75%, 80%, 85% or 90% identical to a reference sequence only differs from the reference sequence by conservative substitutions.

TABLE 1

| Conservative | |
|---|---|
| substitutions | Type of Amino Acid |
| Ala, Val, Leu, Ile, | Amino acids with aliphatic hydrophobic side chains |
| Met, Pro, Phe, Trp | |
| Ser, Tyr, Asn, | Amino acids with uncharged but polar side chains |
| Gln, Cys | |
| Asp, Glu | Amino acids with acidic side chains |
| Lys, Arg, His | Amino acids with basic side chains |
| Gly | Neutral side chain |

In another preferred embodiment, the peptide consisting of an amino acid sequence at least 70%, 75%, 80%, 85% or 90% identical to a reference sequence corresponds to a naturally-occurring allelic variant of the reference sequence.

In still another preferred embodiment, the peptide consisting of an amino acid sequence at least 70%, 75%, 80%, 85% or 90% identical to a reference sequence corresponds to a homologous sequence derived from another non-human mammalian species than the reference sequence.

In a preferred embodiment, the peptide consisting of an amino acid sequence at least 70%, 75%, 80%, 85% or 90% identical to a reference sequence differs from the reference sequence by conservative substitutions and/or corresponds to a homologous sequence derived from another non-human mammalian species than the reference sequence.

By the expression “a peptide consisting of a retro-inverso sequence of peptide (i), (ii) or (iii)”, it is herein meant a peptide that differs from the peptide (i), (ii) or (iii) in that its amino acids are in the reverse order by comparison to the sequence of peptide (i), (ii) or (iii), respectively, and consist of D-amino acids instead of the naturally-occurring L-amino acids.

D-enantiomers of amino acids (also called D-amino acids) are referred to by the same letter as their corresponding L-enantiomer (also called L-amino acid), but in lower case. Thus, for example, the L-enantiomer of arginine is referred to as ‘R’, while the D-enantiomer is referred to as ‘r’.

Preferably, the peptide is soluble in an organic or nonorganic solvent.

In a preferred embodiment, the peptide is soluble in water. More particularly, the peptide is preferably soluble in water and/or in aqueous buffer such as NaCl 9 g/L, PBS, Tris or Tris-phosphate. The solubility in water and aqueous buffers is particularly advantageous on the pharmacological point of view. Thanks to such solubility, the peptide may be dissolved in an aqueous solution, for example at a concentration equal to, at least of or at most of 1 micromolar, 10 micromolar, 50 micromolar, 100 micromolar, 500 micromolar, 1 mM, 50 mM or 100 mM.

A peptide of the invention that is readily soluble in water may be obtained by the presence of at least one charged amino acid (preferably arginine (R) and/or lysine (K)), wherein said charged amino acid is not comprised between two hydrophobic residues.

Thus, in a preferred embodiment, the peptide according to the invention comprises at least one charged amino acid, preferably arginine and/or lysine, wherein said charged amino acid is not comprised between two hydrophobic residues.

In a more preferred embodiment, said charged amino acid is located either at the N- or C-terminal end of the sequence.

For example, the sequence of a preferred peptide according to the invention begins with the motif RV (for example instead of VRV).

In a preferred embodiment, the peptide is resistant to peptidase, in particular to eukaryote peptidase.

By “resistant to peptidase”, it is herein meant that the peptide remains undigested, as determined by reverse phase-high-performance liquid chromatography (RP-HPLC) and mass spectroscopy (MS), upon incubation at 37° C. with mammalian serum or injection in a living laboratory animal. Laboratory tests aimed at evaluating serum stability of the peptides are well standardized (see for example Jenssen and Aspmo, 2008, Methods Mol Biol 494, 177-186). Highly peptidase-resistant peptides are those that remains undigested for up to 70% of their original mass and/or displaying a half life longer than 240 minutes in the presence of proteolytic enzymes (see for example Kumarasinghe and Hruby, 2015, In Peptide Chemistry and Drug Design, B. M. Dunn, ed. (Hoboken, N.J.: Wiley), pp. 247-266).

The peptide is preferably resistant to peptidases present in blood, such as soluble peptidases or peptidases present on cell surface.

The peptide may also comprise at least one or at least one further chemical modification, preferably to improve its stability and/or bioavailability.

Such chemical modifications generally aim at obtaining peptides with increased protection of the peptides against enzymatic degradation in vivo and/or increased capacity to cross membrane barriers, thus increasing its half-life and/or maintaining or improving its biological activity. Any chemical modification known in the art can be employed according to the present invention.

The peptide may comprise at least one artificial amino acid, said artificial amino acid being preferably selected from the group consisting of a D-enantiomer amino acid, a beta-methyl amino acid, a alpha-substituted alpha-amino acid and an amino acid analog.

By “beta-methyl amino acid”, it is herein meant a derivative of the amino acid alanine with an aminomethyl group on the side chain. This non-proteinogenic amino acid is classified as a polar base.

By “alpha-substituted alpha-amino acid”, it is herein meant that the group on the alpha carbon of an L-amino acid (NH2) has been changed to another, non proteinaceous group, such as a methyl-, aryl- or acyl- group.

By “amino acid analog”, it is herein meant any other artificial analog of a natural amino acid.

The peptide may thus comprise at least one amino acid in the D-enantiomer form. For example, 1, 2, 3, 4, 5, 6, 7, 8, 9, 10, 11, 12, 13, 14 or 15 of the amino acids of the peptide defined above may be in the D-enantiomer form.

In one embodiment, the peptide consists of D-amino acids.

The peptide may also comprise an inverted sequence, namely an inversion of the amino acid chain (from the C-terminal end to the N-terminal end). The entire amino acid sequence of the peptide may be inverted, or a portion of the amino acid sequence may be inverted. For example, a consecutive sequence of 2, 3, 4, 5, 6, 7, 8, 9, 10, 11, 12, 13, 14 or 15 amino acids may be inverted. Reference herein to ‘inverted’ amino acids refers to inversion of the sequence of consecutive amino acids in the sequence.

Other chemical modifications include, but are not limited to:

modifications to the N-terminal and/or C-terminal ends of the peptides such as e.g. N-terminal acylation (preferably acetylation) or deamination, or modification of the C-terminal carboxyl group into an amide or an alcohol group;

modifications at the amide bond between two amino acids: acylation (preferably acetylation) or alkylation (preferably methylation) at the nitrogen atom or the alpha carbon of the amide bond linking two amino acids;

modifications at the alpha carbon of the amide bond linking two amino acids such as e.g. acylation (preferably acetylation) or alkylation (preferably methylation) at the alpha carbon of the amide bond linking two amino acids;

retro-inversions in which one or more naturally-occurring amino acids (L-enantiomer) are replaced with the corresponding D-enantiomers, together with an inversion of the amino acid chain (from the C-terminal end to the N-terminal end);

azapeptides, in which one or more alpha carbons are replaced with nitrogen atoms; and/or

betapeptides, in which the amino group of one or more amino acid is bonded to the β carbon rather than the a carbon.

The peptide includes amino acids modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini, it will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from natural post-translational processes or may be made by synthetic methods. Modifications include acetylation, acylation, ADP-ribosylation, araidation, covalent attachment of flavin, covalent attachment of a heme moiety, covalent attachment of a nucleotide or nucleotide derivative, covalent attachment of a lipid or lipid derivative, covalent attachment of phosphatidyl inositol, cross-linking, cyclization, disulfide bond formation, demethylation, formation of covalent cross-links, formation of cystine, formation of pyroglutarnate, formylation, gamma-carboxy!ation, glycosylation, GP'I anchor formation, hydroxylation, iodination, mefhylation, myristoylation, oxidation, proteolytic processing, phosphorylation, prenylation, racemization, selenoyiation, sulfation, transfer-RNA mediated addition of amino acids to proteins such as arginylation, and ubiquitination.

In a preferred embodiment of the invention, the peptide is selected in the group consisting of:

(i) a peptide consisting of a fragment of 3 to 15 amino acids of the sequence defined by amino acids 579 to 601 of sequence SEQ ID NO: 1, said fragment comprising the amino acids 579 to 581, the amino acids 589 to 591, the amino acids 599 to 601 and/or the amino acids 593 to 595 of SEQ ID NO: 1,

(ii) a peptide consisting of a fragment of 3 to 15 amino acids of a sequence corresponding to the amino acids 579 to 601 of sequence SEQ ID NO: 1 in a non-human mammalian CD31, for example a fragment of 3 to 15 amino acids of the sequence defined by amino acids 568 to 590 of sequence SEQ ID NO: 9, said fragment preferably comprising the amino acids 568 to 570, the amino acids 578 to 580, the amino acids 588 to 590 and/or the amino acids 582 to 584 of SEQ ID NO: 9,

(iii) a peptide of 3 to 15 amino acids consisting of a sequence at least 70% identical, preferably at least 75% identical, preferably at least 80% identical, more preferably at least 85% identical, still more preferably at least 90% identical to the sequence of peptide (i),

(iv) a peptide consisting of a retro-inverso sequence of peptide (i), (ii) or (iii), and

(v) the peptide (i), (ii), (iii) or (iv) comprising at least one or at least one further chemical modification.

Such peptide has, for example, a sequence selected from the group consisting of:

SSTLAVRVFLAPWKK (SEQ ID NO: 13, amino acids 576 to 590 of SEQ ID NO: 9), STLAVRVFLAPWKK (SEQ ID NO: 14, amino acids 577 to 590 of SEQ ID NO: 9), TLAVRVFLAPWKK (SEQ ID NO: 15, amino acids 578 to 590 of SEQ ID NO: 9), LAVRVFLAPWKK (SEQ ID NO: 16, amino acids 579 to 590 of SEQ ID NO: 9), AVRVFLAPWKK (SEQ ID NO: 17, amino acids 580 to 590 of SEQ ID NO: 9), VRVFLAPWKK (SEQ ID NO: 3, amino acids 581 to 590 of SEQ ID NO: 9), RVFLAPWKK (SEQ ID NO: 18, amino acids 582 to 590 of SEQ ID NO: 9), VFLAPWKK (SEQ ID NO: 19, amino acids 583 to 590 of SEQ ID NO: 9), FLAPWKK (SEQ ID NO: 20, amino acids 584 to 590 of SEQ ID NO: 9), LAPWKK (SEQ ID NO: 2, amino acids 585 to 590 of SEQ ID NO: 9), APWKK (SEQ ID NO: 21, amino acids 586 to 590 of SEQ ID NO: 9), PWKK (SEQ ID NO: 22, amino acids 587 to 590 of SEQ ID NO: 9), WKK (amino acids 588 to 590 of SEQ ID NO: 9), SKILTVRVILAPWKK (SEQ ID NO: 23, amino acids 587 to 601 of SEQ ID NO: 1), KILTVRVILAPWKK (SEQ ID NO: 24, amino acids 588 to 601 of SEQ ID NO: 1), ILTVRVILAPWKK (SEQ ID NO: 25, amino acids 589 to 601 of SEQ ID NO: 1), LTVRVILAPWKK (SEQ ID NO: 26, amino acids 590 to 601 of SEQ ID NO: 1), TVRVILAPWKK (SEQ ID NO: 27, amino acids 591 to 601 of SEQ ID NO: 1), VRVILAPWKK (SEQ ID NO: 4, amino acids 592 to 601 of SEQ ID NO: 1), RVILAPWKK (SEQ ID NO: 28, amino acids 593 to 601 of SEQ ID NO: 1), VILAPWKK (SEQ ID NO: 29, amino acids 594 to 601 of SEQ ID NO: 1), ILAPWKK (SEQ ID NO: 30, amino acids 595 to 601 of SEQ ID NO: 1), SSMRTSPRSSTLAVR (SEQ ID NO: 31, amino acids 568 to 582 of SEQ ID NO: 9), SSMRTSPRSSTLAV (SEQ ID NO: 32, amino acids 568 to 581 of SEQ ID NO: 9), SSMRTSPRSSTLA (SEQ ID NO: 33, amino acids 568 to 580 of SEQ ID NO: 9), SSMRTSPRSSTL (SEQ ID NO: 34, amino acids 568 to 579 of SEQ ID NO: 9), SSMRTSPRSST (SEQ ID NO: 35, amino acids 568 to 578 of SEQ ID NO: 9), SSMRTSPRSS (SEQ ID NO: 36, amino acids 568 to 577 of SEQ ID NO: 9), SSMRTSPRS (SEQ ID NO: 37, amino acids 568 to 576 of SEQ ID NO: 9), SSMRTSPR (SEQ ID NO: 38, amino acids 568 to 575 of SEQ ID NO: 9), SSMRTSP (SEQ ID NO: 39, amino acids 568 to 574 of SEQ ID NO: 9), SSMRTS (SEQ ID NO: 40, amino acids 568 to 573 of SEQ ID NO: 9), SSMRT (SEQ ID NO: 41, amino acids 568 to 572 of SEQ ID NO: 9), SSMR (SEQ ID NO: 42, amino acids 568 to 571 of SEQ ID NO: 9), SSM (amino acids 568 to 570 of SEQ ID NO: 9), NHASSVPRSKILTVR (SEQ ID NO: 43, amino acids 579 to 593 of SEQ ID NO: 1), NHASSVPRSKILTV (SEQ ID NO: 44, amino acids 579 to 592 of SEQ ID NO: 1), NHASSVPRSKILT (SEQ ID NO: 45, amino acids 579 to 591 of SEQ ID NO: 1), NHASSVPRSKIL (SEQ ID NO: 46, amino acids 579 to 590 of SEQ ID NO: 1), NHASSVPRSKI (SEQ ID NO: 47, amino acids 579 to 589 of SEQ ID NO: 1), NHASSVPRSK (SEQ ID NO: 48, amino acids 579 to 588 of SEQ ID NO: 1), NHASSVPRS (SEQ ID NO: 49, amino acids 579 to 587 of SEQ ID NO: 1), NHASSVPR (SEQ ID NO: 50, amino acids 579 to 586 of SEQ ID NO: 1), NHASSVP (SEQ ID NO: 51, amino acids 579 to 585 of SEQ ID NO: 1), NHASSV (SEQ ID NO: 52, amino acids 579 to 584 of SEQ ID NO: 1), NHASS (SEQ ID NO: 53, amino acids 579 to 583 of SEQ ID NO: 1), NHAS (SEQ ID NO: 54, amino acids 579 to 582 of SEQ ID NO: 1), NHA (amino acids 579 to 581 of SEQ ID NO: 1), TSPRSSTLAVRVFLA (SEQ ID NO: 55, amino acids 572 to 586 of SEQ ID NO: 9), SPRSSTLAVRVFL (SEQ ID NO: 56, amino acids 573 to 585 of SEQ ID NO: 9), PRSSTLAVRVF (SEQ ID NO: 57, amino acids 574 to 584 of SEQ ID NO: 9), RSSTLAVRV (SEQ ID NO: 58, amino acids 575 to 583 of SEQ ID NO: 9), SSTLAVR (SEQ ID NO: 59, amino acids 576 to 582 of SEQ ID NO: 9), STLAV (SEQ ID NO: 60, amino acids 577 to 581 of SEQ ID NO: 9), TLA (amino acids 578 to 580 of SEQ ID NO: 9), SVPRSKILTVRVILA (SEQ ID NO: 61, amino acids 583 to 597 of SEQ ID NO: 1), VPRSKILTVRVIL (SEQ ID NO: 62, amino acids 584 to 596 of SEQ ID NO: 1), PRSKILTVRVI (SEQ ID NO: 63, amino acids 585 to 595 of SEQ ID NO: 1), RSKILTVRV (SEQ ID NO: 64, amino acids 586 to 594 of SEQ ID NO: 1), SKILTVR (SEQ ID NO: 65, amino acids 587 to 593 of SEQ ID NO: 1), KILTV (SEQ ID NO: 66, amino acids 588 to 562 of SEQ ID NO: 1), ILT (amino acids 589 to 591 of SEQ ID NO: 1), RVF (amino acids 582 to 584 of SEQ ID NO: 9), RVFL (SEQ ID NO: 67, amino acids 582 to 585 of SEQ ID NO: 9), RVFLA (SEQ ID NO: 68, amino acids 582 to 586 of SEQ ID NO: 9), RVFLAP (SEQ ID NO: 69, amino acids 582 to 587 of SEQ ID NO: 9), RVFLAPW (SEQ ID NO: 70, amino acids 582 to 588 of SEQ ID NO: 9), RVFLAPWK (SEQ ID NO: 5, amino acids 582 to 589 of SEQ ID NO: 9), RVI (amino acids 593 to 595 of SEQ ID NO: 1), RVIL (SEQ ID NO: 71, amino acids 593 to 596 of SEQ ID NO: 1), RVILA (SEQ ID NO: 72, amino acids 593 to 597 of SEQ ID NO: 1), RVILAP (SEQ ID NO: 73, amino acids 593 to 598 of SEQ ID NO: 1), RVILAPW (SEQ ID NO: 74, amino acids 593 to 599 of SEQ ID NO: 1), RVILAPWK (SEQ ID NO: 7, amino acids 593 to 600 of SEQ ID NO: 1).

In a more preferred embodiment of the invention, the peptide is selected in the group consisting of:

(i) a peptide consisting of a fragment of 3 to 15 amino acids of the sequence defined by amino acids 579 to 601 of sequence SEQ ID NO: 1, said fragment comprising the amino acids 579 to 582, the amino acids 588 to 592, the amino acids 598 to 601 and/or the amino acids 593 to 595 of SEQ ID NO: 1,

(ii) a peptide consisting of a fragment of 3 to 15 amino acids of a sequence corresponding to the amino acids 579 to 601 of sequence SEQ ID NO: 1 in a non-human mammalian CD31, for example a fragment of 3 to 15 amino acids of the sequence defined by amino acids 568 to 590 of sequence SEQ ID NO: 9, said fragment preferably comprising the amino acids 568 to 571, the amino acids 578 to 580 the amino acids 587 to 590 and/or the amino acids 582 to 584 of SEQ ID NO: 9,

(iii) a peptide of 3 to 15 amino acids consisting of a sequence at least 70% identical, preferably at least 75% identical, preferably at least 80% identical, more preferably at least 85% identical, still more preferably at least 90% identical to the sequence of peptide (i),

(iv) a peptide consisting of a retro-inverso sequence of peptide (i), (ii) or (iii), and

(v) the peptide (i), (ii), (iii) or (iv) comprising at least one or at least one further chemical modification.

In a preferred embodiment, the peptide is an 8 amino-acid fragment comprising inversions and/or at least one unnatural amino acid, such as at least one D-amino acids. Such peptides indeed retain the activity of the original peptide or even demonstrate improved activity. Incorporation of unnatural amino acids in peptides intended for therapeutic use is of utility in increasing the stability of the peptide, in particular in vivo stability.

In another preferred embodiment of the invention, the peptide is selected in the group consisting of a peptide of sequence SEQ ID NO: 2, a peptide of sequence SEQ ID NO: 3, a peptide of sequence SEQ ID NO: 4, a peptide of sequence SEQ ID NO: 5, a peptide of sequence SEQ ID NO: 6 consisting of D-enantiomer amino acids, a peptide of sequence SEQ ID NO: 7 and a peptide of sequence SEQ ID NO: 8 consisting of D-enantiomer amino acids.

A more preferred peptide is a peptide of sequence SEQ ID NO: 5 or a peptide of sequence SEQ ID NO: 6 consisting of D-enantiomer amino acids.

The peptide may be prepared by any well-known procedure in the art, such as chemical synthesis, for example solid phase synthesis or liquid phase synthesis, or genetic engineering. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the amino groups of the peptides are released, the peptide chain is thus extended in this manner. After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

Solid phase synthesis methods are largely classified by the tBoc method and the Fmoc method, depending on the type of protective group used. Typically used protective groups include tBoe (t-butoxycarbonyl), Cl-Z (2-chlorobenzyloxycarbonyl), Br-Z (2-bromobenzyloyycarbonyl), BzI (benzyl), Fmoc (9-fluorenylmcthoxycarbonyl), Mbh (4, 4′-dimethoxydibenzhydryl), Mtr (4-methoxy-2, 3, 6-trimethylbenzenesulphonyl), Trt (trityl), Tos (tosyl), Z (benzyloxycarbonyl) and Clz-Bzl (2, 6-dichlorobenzyl) for the amino groups; NO2 (nitro) and Pmc (2,2, 5,7, 8-pentamethylchromane-6-sulphonyl) for the guanidino groups); and tBu (t-butyl) for the hydroxyl groups).

Alternatively, the CD31 peptide may be synthesized using recombinant techniques.

The method of producing the peptide may optionally comprise the steps of purifying said peptide, chemically modifying said peptide, and/or formulating said peptide into a pharmaceutical composition.

### Peptidomimetic

In an embodiment, the CD31^{shed} agonist is a peptidomimetic of a peptide as defined above in the section “Peptide used as CD31^{shed} agonist”, i.e. a compound that mimics said peptide.

A «peptidomimetic» is a compound consisting of non-peptidic structural elements that mimics a given peptide, thereby conferring to said compound a biological activity equal to or similar to said peptide.

The peptidomimetic is preferably soluble in an organic or nonorganic solvent.

As the peptide according to the invention, the peptidomimetic is preferably soluble in water.

Methods for designing and synthesizing peptidomimetics of a given peptide are well-known in the art and include e.g. those described in Ripka and Rich (Curr. Opin. Chem. Biol. 1998; 2(4):441-52) and in Patch and Barron (Curr. Opin. Chem. Biol. 2002; 6(6):872-7).

A peptidomimetic as defined above may be obtained by at least one chemical modification selected in the group consisting of structural modification of a peptide by using unnatural amino acid(s).

### Pharmaceutical Composition

The CD31^{shed} agonist may be formulated into a pharmaceutical composition. Thus, the invention contemplates a pharmaceutical composition comprising at least one CD31^{shed} agonist and, preferably, a pharmaceutically acceptable vehicle.

The CD31^{shed} agonist is as defined above, in particular in the sections “CD31^{shed} agonist”, “Peptide used as CD31^{shed} agonist” and “peptidomimetic”.

Pharmaceutical compositions comprising at least one CD31^{shed} agonist include all compositions, wherein the CD31^{shed} agonist is contained in an amount effective to achieve the intended purpose, in particular the prevention and/or the treatment of reperfusion injury.

The expression “pharmaceutically acceptable” is meant to encompass any carrier, which preferably does not interfere with the effectiveness of the biological activity of the active ingredient and/or that is preferably not toxic to the host to which is administered.

Pharmaceutically acceptable vehicles can be prepared by any method known by those skilled in the art.

Suitable pharmaceutically acceptable vehicles may comprise excipients and auxiliaries, which facilitate processing of the active compounds into preparations which can be used pharmaceutically. Suitable pharmaceutically acceptable vehicles are described for example in Remington's Pharmaceutical Sciences (Mack Publishing Company, Easton, USA, 1985), which is a standard reference text in this field. Pharmaceutically acceptable vehicles can be routinely selected in accordance with the mode of administration, solubility and stability of the CD31^{shed} agonist. For example, formulations for intravenous administration may include sterile aqueous solutions which may also contain buffers, diluents and other suitable additives. The use of biomaterials and other polymers for drug delivery, as well the different techniques and models to validate a specific mode of administration, are disclosed in literature.

Dosages to be administered depend on the subject to be treated, on the desired effect and the chosen route of administration. It is understood that the dosage administered will be dependent upon the age, sex, health, and weight of the recipient, concurrent treatment, if any, and frequency of treatment, and the nature of the effect desired. The total dose required for each treatment may be administered by multiple doses or in a single dose.

The CD31^{shed} agonist is preferably formulated as liquid (e.g. solution, suspension).

In a preferred embodiment, the pharmaceutical compositions are presented in unit dosage forms to facilitate accurate dosing. The term “unit dosage forms” refers to physically discrete units suitable as unitary dosages for human subjects and other mammals, each unit containing a pre-determined quantity of active material calculated to produce the desired therapeutic effect, in association with a suitable pharmaceutical excipient. Typical unit dosage forms include pre-filled, pre-measured ampoules or syringes of the liquid compositions. In such compositions, the CD31^{shed} agonist is usually a minor component (for example from about 0.1 to about 50% by weight or preferably from about 1 to about 40% by weight), with the remainder being various vehicles or carriers and processing aids helpful for forming the desired dosing form.

Besides the pharmaceutically acceptable vehicle, the pharmaceutical composition can also comprise minor amounts of additive(s), such as stabilizer(s), excipient(s), buffer(s) and/or preservative(s).

The invention further provides kits comprising a pharmaceutical composition comprising a CD31^{shed} agonist as defined above and instructions regarding the mode of administration. These instructions may e.g. indicate the medical indication, the route of administration, the dosage and/or the group of patients to be treated.

### Ischemia

In a preferred embodiment, reperfusion is used for treating ischemia.

Ischemia is a stasis, restriction or arrest of blood supply to tissues.

Ischemia is preferably selected in the group consisting of myocardial infarction, ischemic colitis, mesenteric ischemia, stroke, ischemia of the lower limbs, visceral ischemia resulting from acute hypovolemia, ischemia resulting from inflammatory conditions, ischemia inherent to an extracorporeal circulation blood excluding part of the arterial branches, ischemia inherent to a visceral and/or aortic surgery, cold ischemia, ischemia inherent to warm reperfusion of a graft directed to organ transplantation and their combinations.

Myocardial infarction occurs when the blood flow in the heart muscle is insufficient. Myocardial infarction may be asymptomatic or cause chest pain, known as angina pectoris.

Both large and small bowel can be affected by ischemia.

Ischemia of the large intestine is referred to as ischemic colitis.

Mesenteric ischemia corresponds to ischemia of the small bowel.

Stroke is a brain ischemia that may be acute or chronic. Acute ischemic stroke is a neurologic emergency that may be reversible if treated rapidly. Chronic ischemia of the brain may result in a form of dementia.

Ischemia of the lower limbs is a lack of blood flow in at least one limb.

Visceral ischemia resulting from acute hypovolemia is a lack of visceral perfusion due to the redistribution of the blood to other tissues (brain, heart) in case of reduced blood content (for example after an hemorrhage) and/or blood pressure (for example due to an abrupt dilatation of the vascular bed as it occurs during septic, cytokinic, or cardiogenic chock).

Artificial ischemia is an ischemia inherent to a medical procedure.

Ischemia inherent to a visceral and/or aortic surgery is an ischemia resulting from an arrest of the blood circulation performed by a surgeon, for example by a transient clamping of a blood vessel upstream an organ.

Ischemia of a graft occurs once a tissue or organ to be transplanted is removed from a donor. The tissue or organ to be transplanted is generally washed and chilled, for example at 4° C., to reduce the metabolic damage resulting from ischemia. The ischemia of a cooled down tissue or organ is called cold ischemia.

Cold ischemia refers to the ischemic damage that occurs in an organ that is explanted and hence disconnected from the circulation of the donor.

Warm reperfusion indicates the reestablishment of the circulation (reperfusion) inherent to the graft of the transplanted organ in the recipient.

The expression “cold ischemia time” herein means the time between the chilling of a tissue or organ after its blood supply has been reduced or cut off and the time it is warmed by having its blood supply restored in vivo.

Ischemia resulting from inflammatory conditions is for example an ischemia of the bowel, which may for example occur in Crohn disease and/or ulcerative colitis.

In one embodiment of the invention, reperfusion is associated with the increase of soluble truncated CD31 in biological fluids (including plasma), and thus to a CD31-T lymphocyte phenotype.

As used herein, the term “CD31-T lymphocyte phenotype” is used interchangeably with the term “CD31^{shed} lymphocyte phenotype”. These terms refer to the phenotype of an individual having apparently lost CD31 on its circulating T cells when conventional prior art methods for detecting CD31, e.g. such as those described in Stockinger et al. (Immunology, 1992, 75(1):53-8), Demeure et al. (Immunology, 1996, 88(1):110-5), Caligiuri et al. (Arterioscler Thromb Vasc Biol, 2005, 25(8):1659-64) or Caligiuri et al. (Arterioscler Thromb Vasc Biol, 2006, 26(3):618-23) are used. In such methods, the antibody used for detecting CD31 binds to an epitope located on any one of the 1st to the 5th extracellular Ig-like domains (see document U.S. Pat. No. 8,951,743).

Preferably, individuals having a CD31-T lymphocyte phenotype particularly have at least 50%, 60%, 65%, 70%, 75%, 80%, 90% or 95% of their circulating T lymphocytes that are CD31^{shed} lymphocytes. In order to calculate this percentage, either the plasma concentration of T-cell-derived truncated CD31 or the frequency of CD31-T lymphocytes, compared to CD31+ T lymphocytes, may be measured.

### Subject To Be Treated

A subject in need to be treated for the prevention and/or treatment of reperfusion injury may be an individual or a non-human mammal.

The terms “individual” or “patient” are used interchangeably and refer to a human being.

Said human being may be of any age, for example an infant, child, adolescent, adult, elderly people.

A non-human mammal is preferably a mouse, rat, cat, dog, rabbit or primate.

The subject preferably suffers from ischemia, being treated or not by reperfusion.

### Prevention and/or Treatment of Reperfusion Injury

By the expression “treatment of reperfusion injury”, it is herein meant to eliminate or reduce organ damage caused by reperfusion and/or to stop or slow down the progression of organ damage caused by reperfusion, in particular after ischemia, in particular in a subject under reperfusion who already suffers or do not suffer from organ damage.

Desirable effects of treatment include:
stopping or slowing down the progression of necrosis, for example decreasing the size of the necrotic area(s), in particular in the ischemic tissue or organ during warm reperfusion (for example in heart, brain, intestine, kidneys and/or limbs),
increasing the chances of organ survival after the reperfusion,
stopping or reducing the lesion(s) of the intestinal epithelium, in particular in terms of length and width of the villus, mitotic index and/or intra-tissue hemorrhage, for example during reperfusion further to a mesenteric ischemia,
stopping or reducing bacterial translocation, in particular in blood, spleen, liver and/or mesenteric ganglions, for example during reperfusion further to a mesenteric ischemia, and/or
increasing the cold ischemia time and/or improving the recovery of a graft, in the case of a tissue or organ transplantation.

By the expression “prevention of reperfusion injury”, it is herein meant to prevent, at least partially, the organ damage in a subject that is likely to be subjected to reperfusion.

Desirable effects of prevention include:
preventing, at least partially, necrosis,
increasing the chances of organ survival after reperfusion,
increasing the time frame/delay for getting a positive benefit/risk ratio for performing reperfusion after prolonged ischemia,
preventing, at least partially, the function of the organ (for example preventing reduced mucus associated with damaged intestinal epithelium, reduced contractile function of the myocardium, reduced motility following cerebral or limb reperfusion),
preventing, at least partially, bacterial translocation, in particular in blood, spleen, liver and/or mesenteric lymph nodes, for example during reperfusion further to a mesenteric ischemia, and/or
increasing the cold ischemia time and/or improving the warm recovery of a graft, in the case of a tissue or organ transplantation.

By «necrosis», it is herein meant the death of one or several cells of a tissue.

Reperfusion, in particular reperfusion for treating ischemia, may be obtained by mechanical revascularization and/or pharmacological reperfusion.

Mechanical revascularization may for example comprise angioplasty and/or thromboaspiration and/or stent implantation and/or endoarterectomy and/or bypass surgery.

Pharmacological reperfusion may for example comprise the administration of at least one fibrinolytic agent and/or antiplatelet/anticoagulation drug(s) and/or vasodilator drug(s).

Fibrinolytic (thrombolytic) agents are activators of fibrin-bound plasminogen; their action consists in converting the zymogen plasminogen to the active enzyme plasmin, which degrades fibrin. Non-limitative examples of fibrinolytic agents available for clinical use are: the physiologic tissue-type plasminogen activator (t-PA) and urokinase-type plasminogen activator (u-PA)—either in a single chain (scu-PA, prourokinase) or a two-chain (tcu-PA, urokinase) form, and the bacterial activator plasminogen streptokinase or its anisoylated complex with plasminogen (APSAC).

### CD31^{shed} Agonist for Use in the Prevention and/or Treatment of Reperfusion Injury

The present invention particularly relates to a CD31^{shed} agonist for use in the prevention and/or treatment of reperfusion injury.

The prevention and/or treatment of reperfusion injury is particularly as defined above. The CD31^{shed} agonist is as defined above, in particular in the sections “CD31^{shed} agonist”, “Peptide used as CD31^{shed} agonist” and “peptidomimetic”.

The CD31^{shed} agonist may be provided in the form of a pharmaceutical composition. Said pharmaceutical composition is particularly as defined above in the section “pharmaceutical composition”.

The present invention thus relates to a CD31^{shed} agonist or pharmaceutical composition as defined above for use in the prevention and/or treatment of reperfusion injury in a subject in need thereof.

The subject in need thereof is as defined above in the section “Subject to be treated”.

Said subject in need thereof preferably suffers from ischemia.

Ischemia is as defined above in section “Ischemia”.

The present invention thus particularly relates to a CD31^{shed} agonist or pharmaceutical composition for use as defined above, wherein reperfusion is used for treating ischemia.

The present invention particularly relates to a CD31^{shed} agonist or pharmaceutical composition for use as defined above, wherein said CD31^{shed} agonist:
stops or slows down the progression of necrosis, for example decreases the size of the necrotic area(s), in particular in the ischemic tissue or organ during warm reperfusion (for example in heart, brain, intestine, kidneys and/or limbs),
increases the chances of organ survival after a reperfusion,
stops or inhibits the lesion(s) of the intestinal epithelium, in particular in terms of length and width of the villus, mitotic index and/or intra-tissue hemorrhage, for example during reperfusion further to a mesenteric ischemia,
stops or reduces bacterial translocation, in particular in blood, liver, spleen and/or mesenteric ganglions, for example during a reperfusion further to a mesenteric ischemia, and/or
increases the cold ischemia time and/or improve the warm recovery of a graft, in the case of a tissue or organ transplantation.

The CD31^{shed} agonist or pharmaceutical composition is preferably used or administered prior to and/or during reperfusion, preferably during the entire ischemia-reperfusion period, if possible.

In the case of ischemia caused by elective visceral and/or aortic surgery, the CD31^{shed} agonist or pharmaceutical composition is preferably used or administered prior to and during reperfusion, for example during the ischemia-reperfusion period, more preferably both prior to ischemia and during the ischemia-reperfusion period.

Ischemia begins when the blood circulation is stopped in a blood vessel. For example, in the case of an artificial ischemia, the blood circulation is stopped when the blood vessel is clamped.

Reperfusion begins when the blood circulation is restored. The damage caused by reperfusion generally occurs within 48 hours, preferably 24 hours.

The term “prior to” in the expressions “prior to ischemia” or “prior to reperfusion” for example means that the CD31^{shed} agonist is used or administered at most 1 hour before, preferably at most 30 minutes before, more preferably at most 20 minutes before, more preferably at most 10 minutes before, for example 10 minutes before, 5 minutes before, 2 minutes before or immediately before.

In the case of a non-scheduled ischemia, the CD31^{shed} agonist or pharmaceutical composition is used or administered as soon as possible, for example prior to and/or during reperfusion, preferably prior to and during reperfusion.

The CD31^{shed} agonist or pharmaceutical composition is preferably used or administered intravenously. Intravenous administration allows obtaining quickly an effective amount of CD31^{shed} agonist at the location of reperfusion, in particular in the ischemia area, and/or to maintain an effective amount of CD31^{shed} agonist by intravenous infusion.

In a preferred embodiment, the CD31^{shed} agonist or pharmaceutical composition is used or administered continuously during reperfusion, preferably after the administration of an initial bolus of CD31^{shed} agonist as a single dose.

For example, a first bolus of CD31^{shed} agonist is used or administered as a single dose, followed by a second bolus of said CD31^{shed} agonist used or administered continuously. In one embodiment of the invention, said first and second bolus may comprise the same quantity of CD31^{shed} agonist.

The bolus for administration as a single dose or continuously may comprise from 2.5 to 30 mg of CD31^{shed} agonist/kg of the subject, preferably from 2.5 to 20 mg of CD31^{shed} agonist/kg of the subject, more preferably from 2.5 to 10 mg of CD31^{shed} agonist/kg of the subject. The CD31^{shed} agonist or pharmaceutical composition is preferably used or administered during a short period of time, for example at most for 48 hours, more preferably at most for 24 h.

In the case of transplantation, the CD31^{shed} agonist or pharmaceutical composition is preferably used or administered to the subject to be treated, i.e. to the receiver, prior to grafting the explanted organ (which will then incur immediate, warm reperfusion). In an advantageous embodiment of the invention, the CD31^{shed} agonist is also used or administered to the donor, prior to the removal of the tissue or organ to be transplanted, for example 5 to 10 minutes before the removal of the tissue or organ and/or the removed organ or tissue is continuously perfused during the transportation and/or washing period.

### Method for Preventing and/or Treating Reperfusion Injury

The invention is also directed to a method for preventing and/or treating reperfusion injury in a subject in need thereof, said method comprising a step of administering an effective amount of a CD31^{shed} agonist to said subject.

The prevention and/or treatment of reperfusion injury is particularly as defined above.

The CD31^{shed} agonist is as defined above, in particular in the sections “CD31^{shed} agonist”, “Peptide used as CD31^{shed} agonist” and “peptidomimetic”.

The CD31^{shed} agonist may be provided in the form of a pharmaceutical composition. Said pharmaceutical composition is particularly as defined above in the section “pharmaceutical composition”.

The subject in need thereof is as defined above in the section “Subject to be treated”.

Said subject in need thereof preferably suffers from ischemia.

Ischemia is as defined above in section “Ischemia”.

The present invention thus particularly relates to a method for preventing and/or treating reperfusion injury as defined above, wherein reperfusion is used for treating ischemia.

By “effective amount” or “therapeutically effective amount”, it is herein meant an amount sufficient to achieve a concentration of the CD31^{shed} agonist, which is capable of preventing and/or treating reperfusion injury. Such effective amounts can be routinely determined by those of skilled in the art. The amount of the compound actually administered will typically be determined by a physician, in the light of the relevant circumstances, including the condition to be treated, the chosen route of administration, the actual compound administered, the age, sex, weight, and response of the individual patient, the severity of the patient's symptoms, and the like. It will also be appreciated by those of skilled in the art that the dosage may be dependent on the stability of the administered CD31^{shed} agonist, in particular in the case of a peptide.

The effective amount may vary according to the drug or prodrug with which the CD31^{shed} agonist may be co-administered.

A therapeutically effective amount encompasses an amount in which any toxic or detrimental effects of the CD31^{shed} agonist are outweighed by the therapeutically beneficial effects. A therapeutically effective amount also encompasses an amount sufficient to confer benefit, e.g., clinical benefit.

The dosage, mode of administration and effects of the CD31^{shed} agonist are particularly as defined above in the section “CD31^{shed} agonist for use in the prevention and/or treatment of reperfusion injury”.

The present invention will be further illustrated in view of the following examples and figures.

All references cited herein, including journal articles or abstracts, published or unpublished patent application, issued patents or any other references, are entirely incorporated by reference herein, including all data, tables, figures and text presented in the cited references.

### BRIEF DESCRIPTION OF THE FIGURES

FIG. 1: Percentage of survival after 45 minutes of ischemia by ligation of the coronary artery, followed by 24 hours of reperfusion is significantly reduced in the absence of CD31. WT: wild mice (90% survival; n=17, 2 dead); CD31 KO: CD31 knockout mice (45% survival, n=9, 4 dead). CHITEST<0.001.

FIG. 2: Effect of P8RI (10 mg/Kg P8RI by subcutaneous injection, 2 minutes prior to reperfusion) on the area at risk and size of myocardial infarction in apolipoprotein E knockout mice (female, 18 week-old) subjected to 45 minutes of ischemia by ligation of the coronary artery, followed by 24 hours of reperfusion. The figure shows the average AAR (percentage of the left ventricle, LV) and MI (expressed both as percentage of the AAR and of the LV). P8RI: mice receiving P8RI (n=5); Control: mice receiving a similar volume of vehicle (PBS) without P8RI (n=5). T-Test.

FIG. 3: Quantification of the free DNA (in ng/ml) in the plasma of rats subjected to ischemia-reperfusion model over time (in hours). The free plasma DNA level increases with time in the control rats (upper line) whereas in the rats treated with the peptide P8RI (middle line) it remains low and similar to sham-operated rats (lower line). MANOVA, repeated measures, group effect: F=0.3010883, p<0.0001.

FIG. 4: Quantification of urinary free DNA (in ng/ml) in the urines of rats subjected to ischemia-reperfusion model over time (in ng/Kg/hour). The free plasma DNA urinary rate is reduced the rats treated with the peptide P8RI (open box) as compared to controls (C, grey box). T-test p=0.0293.

FIG. 5: Quantification of free hemoglobin within the intestinal lumen (ratio, normalized by total protein content) of rats subjected to ischemia-reperfusion model over time. The relative amount of free hemoglobin is decreased in the rats treated with the peptide P8RI (right column) as compared to controls (C, left column). T-test p=0.0293.

FIG. 6: Quantification of free myeloperoxidase MPO in the intestinal mucosa (Direct ELISA, data expressed as arbitrary units—optical density) of rats subjected to ischemia-reperfusion model. The relative amount of MPO is decreased in the rats treated with the peptide P8RI (open box) as compared to controls (C, grey box). Mann Whitney U test p<0.001.

FIG. 7: Quantification of soluble plasma P-selectin in the rats subjected to ischemia-reperfusion model. The concentration of P-selectin (in pg/ml) was decreased in the rats treated with the peptide P8RI (open box) as compared to controls (C, grey box). Mann Whitney U test p=0.0173.

FIG. 8: Quantification of plasma MMP9 in the rats subjected to ischemia-reperfusion model as analyzed over time. The cumulative concentration of MMP9 (area under the curve of the different time points) was decreased in the rats treated with the peptide P8RI (open box) as compared to controls (C, grey box). Mann Whitney U test p=0.0082.

FIG. 9: Quantification of the immunoglobulins within the cerebral tissue in a model of cerebral ischemia. The right/left Ig ratio, i.e. [total Ig in the right hemisphere (subjected to ischemia-reperfusion injury)]/[Total Ig in the left hemisphere (contralateral, non-ischemic)] is shown for each group of mice (P8RI: mice treated with P8RI90 minutes after induction of ischemia; saline: mice treated with saline solution 90 minutes after induction of ischemia; Sham: sham-operated mice).

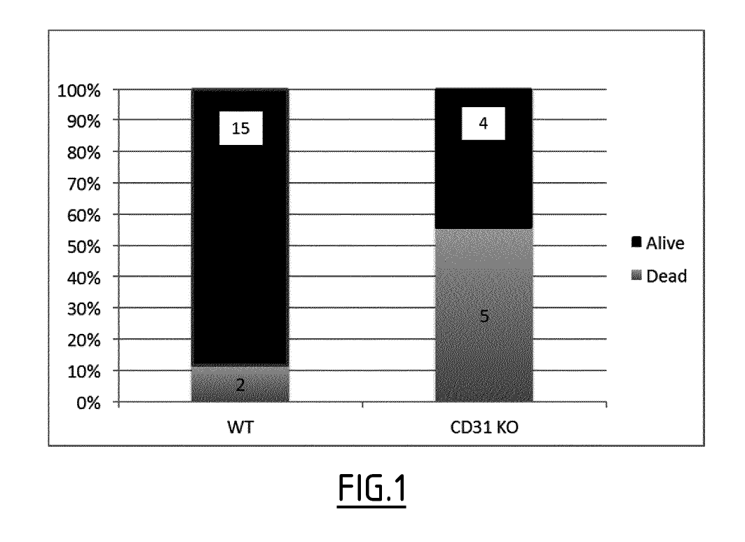

### BRIEF DESCRIPTION OF THE SEQUENCES

SEQ ID NO: 1 corresponds to the sequence of human CD31.

SEQ ID NO: 2 corresponds to the sequence LAPWKK of a 6 amino acid peptide derived from human or murine CD31.

SEQ ID NO: 3 corresponds to the sequence VRVFLAPWKK of a 10 amino acid peptide derived from murine CD31, also called PepReg CD31.

SEQ ID NO: 4 corresponds to the sequence VRVILAPWKK of a 10 amino acid peptide derived from human CD31.

SEQ ID NO: 5 corresponds to the sequence RVFLAPWK of a 8 amino acid peptide derived from murine CD31, also called P8F.

SEQ ID NO: 6 corresponds to the sequence kwpalfvr of a 8 amino acid peptide, also called P8RI, having the inverted sequence of SEQ ID NO: 5 and consisting of D-amino acids.

SEQ ID NO: 7 corresponds to the sequence RVILAPWK of a 8 amino acid peptide derived from human CD31.

SEQ ID NO: 8 corresponds to the sequence kwpalivr of a 8 amino acid peptide having the inverted sequence of SEQ ID NO: 7 and consisting of D-amino acids.

SEQ ID NO: 9 corresponds to the sequence of murine CD31.

SEQ ID NO: 10 corresponds to the sequence of bovine CD31.

SEQ ID NO: 11 corresponds to the sequence of pig CD31.

SEQ ID NO: 12 corresponds to the amino acids 579 to 601 of sequence SEQ ID NO: 1.

### EXAMPLES

### Example 1

### Protective Effect of a CD31^{shed} Agonist in a Model of Myocardial Ischemia-Reperfusion

Material and Methods

In order to reproduce the ischemia-reperfusion injury in the setting of coronary atherosclerosis (as it occurs in patients) were used 18 week-old female apolipoprotein E knockout mice (N=12/group) which typically develop coronary atherosclerosis by the age of 16 week (Caligiuri et al., Atherosclerosis. 1999 August;145(2):301-8). Mice were subjected to surgical ligation of the left main coronary artery, according to the method described by Michael et al. (Am J Physiol. 1995 December;269(6 Pt 2):H2147-54). After 43 minutes of ischemia, the mice received either P8RI (10 mg/kg) or the vehicle (PBS) by subcutaneous injection and 2 minutes later the ischemic area was reperfused by reopening of the ligated coronary artery. The next day, the mice were re-anesthetized, the chest reopened and the coronary ligature re-tied ; the coronary tree was then rinsed with 2 ml of PBS pushed in the coronary tree through retrograde cannulation of the left carotid artery and the non-ischemic area stained by the injection of 50 μl of evan's blue (3% in PBS) in the same cannula. The heart was then quickly removed from the chest and immersed in a saturated KCl solution (to stop the heart in diastole) and rinsed in PBS. The atria and the right ventricle were excised and the left ventricle was cut in 4-5 1 mm-thick slices. The live myocardium within the ischemic area was stained in red by incubating the heart slices in 37° C. warm phosphate buffer containing 1% triphenyltetrazolium chloride for 5 minutes. Each slice was photographed and the area at risk (AAR, negatively stained by the evan's blue) and the necrotic area (MI, negatively stained in red by the TTC solution) were then measured by computer-assisted image analysis.

Results

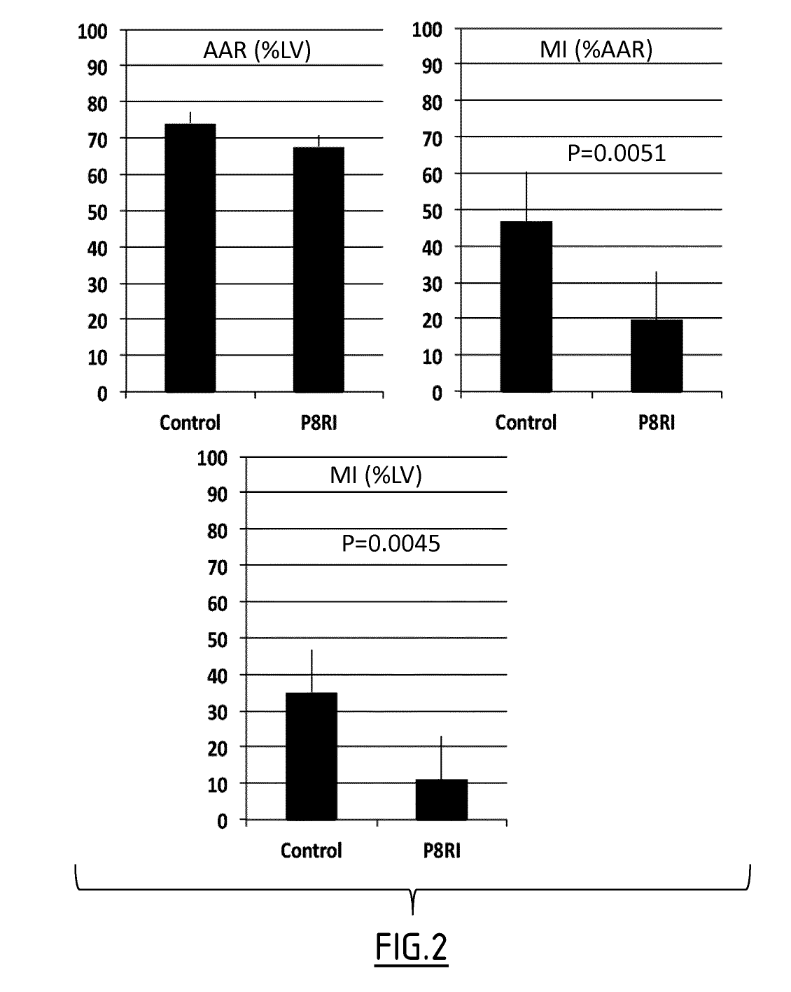

The AAR, expressed as % of the left ventricle (LV) surface and the necrotic area (MI, expressed as percentage of the AAR and of the LV) were calculated on each slice. The data shown in FIG. 2 are expressed as mean±SEM. The AAR was similar in the P8RI and control groups thus confirming that the surgical ligature on the coronary artery was performed in the same way for the two groups. The administration of P8RI2 minutes prior to the reperfusion was significantly effective in reducing the size of myocardial infarction (MI) as percentage of the area at risk (AAR) or of the left ventricle (LV).

### Example 2

### Protective Effect of a CD31^{shed} Agonist in a Model of Cerebral Ischemia-Reperfusion

Materials and Methods

Male C57BU6 mice (8 week old, Charles River, France) were anesthetized with 1.5% isoflurane (Forene, Abbott, Germany) in an air-oxygen mixture under spontaneous respiration.

Focal cerebral ischemia was induced by introducing a silicone-coated 8-0 monofilament into the right common carotid artery and advancing it along the internal carotid artery until the tip occluded the proximal stem of the middle cerebral artery (MCA). Regional cerebral blood flow was monitored by laser Doppler flowmetry (PF5010, Perimed, Sweden) with use of a flexible fiber optic probe fixed to the intact skull above the territory of the right MCA. Rectal temperature was maintained at 37° C. through a thermometer-connected a heating pad. The treatment (P8RI10 mg/Kg body weight or saline, 100 μl subcutaneous bolus) was administered 90 minutes after the induction of ischemia. Two hours after induction of ischemia, the filament was withdrawn to allow reperfusion.

Twenty-four hours after induction of ischemia, mice were deeply anesthetized with isoflurane and transcardially perfused with saline until colorless fluid was obtained from the right atrium at 100 mm Hg. Blood-poor brains (all the blood was washed away from the intracerebral vasculature) were removed quickly, divided into right and left hemispheres, weighted and snap-frozen in liquid nitrogen, and stored at 80° C.

Brain samples were homogenized in a RIPA buffer (150 mM NaCl, 1% Triton, 0.5% sodium deoxycholate, 0.1% SDS, 50mM Tris, pH8+anti-protease cocktail from Roche) at 5 82 l/mg tissue, centrifuged at 16000 g for 20 minutes and supernatant aliquoted and stored at −80° C. until analysis.

In order to assess the effect of P8RI on the permeability of the blood brain barrier, one of the major consequences of ischemia-reperfusion induced local inflammation, the amount of immunoglobulins within the cerebral tissue was measured.

Total immunoglobulins were measured by using a commercially available bead based immunoassay (ProcartaPlex Mouse Antibody Isotyping Panel, Affimetrix cat N° EPX070-20815-901, following the manufacturer's instructions). Mouse immunoglobulin isotypes IgG1, K and L ans IgG2a,K were readily detectable in all samples. Pooled Ig from the three isotypes were used for analysis.

Results

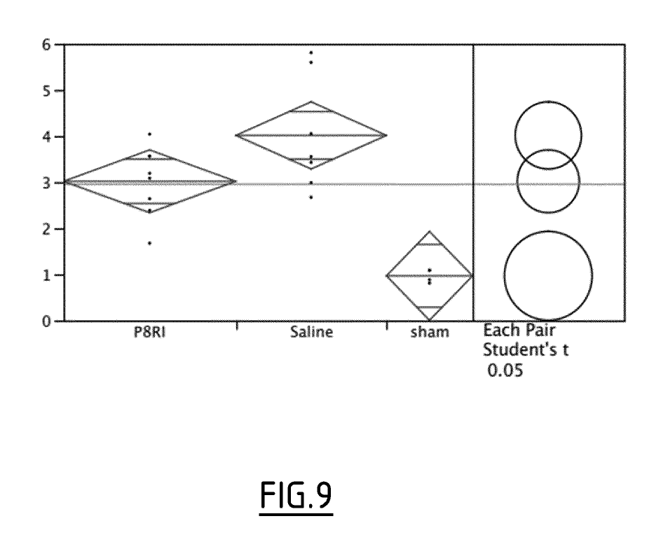

The results are shown in the Table 2 below and FIG. 9. The data are expressed as the right/left Ig ratio:

$$\frac{ \begin{matrix}Total Ig in the right hemisphere \\ ( subjected to ischemia \text{-} reperfusion injury )\end{matrix} }{Total Ig in the left hemisphere ( contralateral , non ischemic )}$$

TABLE 2
Means and Standard Deviations

| | | | Std | Std Error | Lower | Upper |
|---|---|---|---|---|---|---|
| Level | Number | Mean | Deviation | Mean | 95% | 95% |
| P8RI | 8 | 3.01943 | 0.75776 | 0.26791 | 2.3859 | 3.6529 |
| Saline | 7 | 4.01505 | 1.23419 | 0.46648 | 2.8736 | 5.1565 |
| sham | 4 | 0.96883 | 0.14292 | 0.07146 | 0.7414 | 1.1963 |

As shown in the FIG. 9, the right/left Ig ratio was around 1 in sham-operated mice whereas it was consistently increased in mice subjected to ischemia-reperfusion brain injury. On note, the administration of P8RI30 minutes before reperfusion was able to significantly reduce the right/left lg ratio, reflecting a reduced blood brain barrier permeability.

### Example 3

### Protective Effect of a CD31^{shed} Agonist in a Model of Mesenteric Ischemia-Reperfusion

Material and Methods

Rats are anesthetized by urethane I.P. A catheter is introduced in the right jugular vein for venous sampling, treatment administration, and solute perfusion (NaCl 0.9%, 10 μl/H/g b.w.) during all the experimental period. A catheter is introduced in the left internal carotid artery for arterial blood pressure monitoring and another one in the bladder for urine collection. A median laparotomy is then performed and the proximal part of the superior mesenteric artery dissected upper the left renal vein. The aortic ostium of the mesenteric artery is controlled. The intravenous administration of the peptide is initiated 5 minutes before clamping the mesenteric artery and after blood sampling (T0). Then, the mesenteric artery is clamped (total occlusion) for 30 minutes creating mesenteric ischemia. Blood sampling is performed at the end of the mesenteric ischemia period (T0.5). The mesenteric artery is then unclamped and reperfusion is follow-up to 4 hours. New blood samplings are performed each hour (T1.5, 2.5, 3.5, 4.5). The animal is sacrificed at the end of the experimental period after a last blood sampling. Urines are collected and diuresis measured each hour. At sacrifice, the ischemic intestine is sampled. One fragment is extruded in order to measure hemoglobin content. The other one is fixed in paraformaldehyde for histology.

Several intermediary biological criteria are use in order to characterize the ischemia-reperfusion injury and to evaluate the beneficial effect of the CD31^{shed} agonist peptide: plasma and urinary free DNA (Picogreen fluorescent intercalant, Invitrogen), from neutrophil origin, traducing neutrophil activation and death, intra-intestinal lumen hemoglobin content (acid formic reaction, Calbiochem kit), traducing epithelial injury, Myeloperoxidase (MPO) content in the intestinal mucosa, reflecting the local accumulation and activation of neutrophils, plasma soluble p-selectin and MMP9 content, reflecting systemic neutrophil activation, as p-selectin is released by a cleavage from the surface of activated neutrophils (and platelets) and MMP9 is contained in the azurofilic granules of the neutrophils and is released early upon neutrophil degranulation. Finally, the extent of mucin film protection was evaluated by histology of the intestinal wall (Alcian blue staining).

### Results

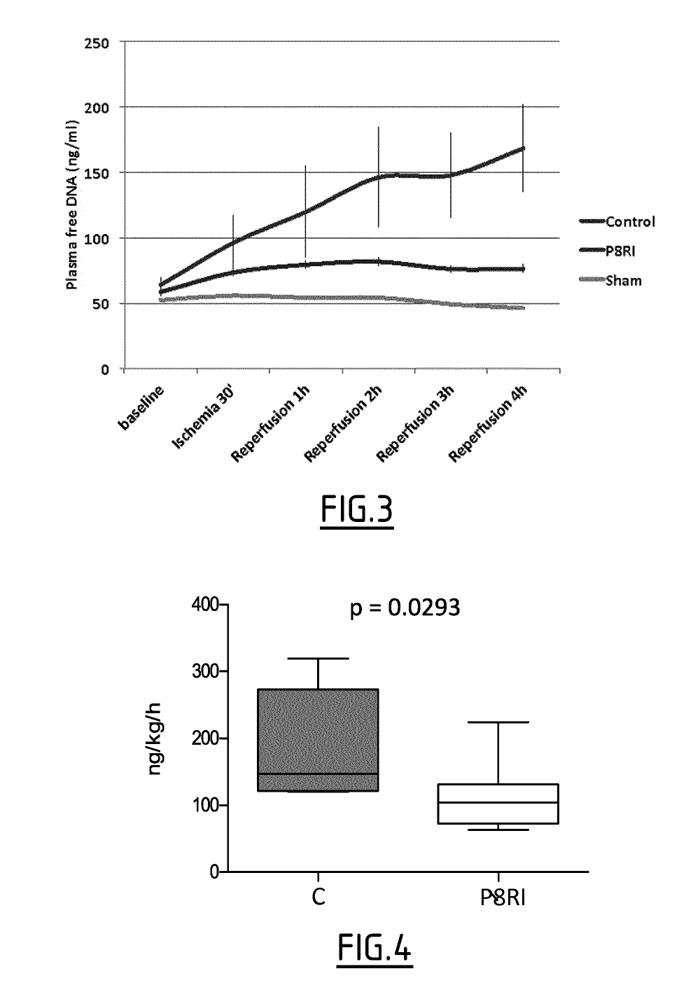

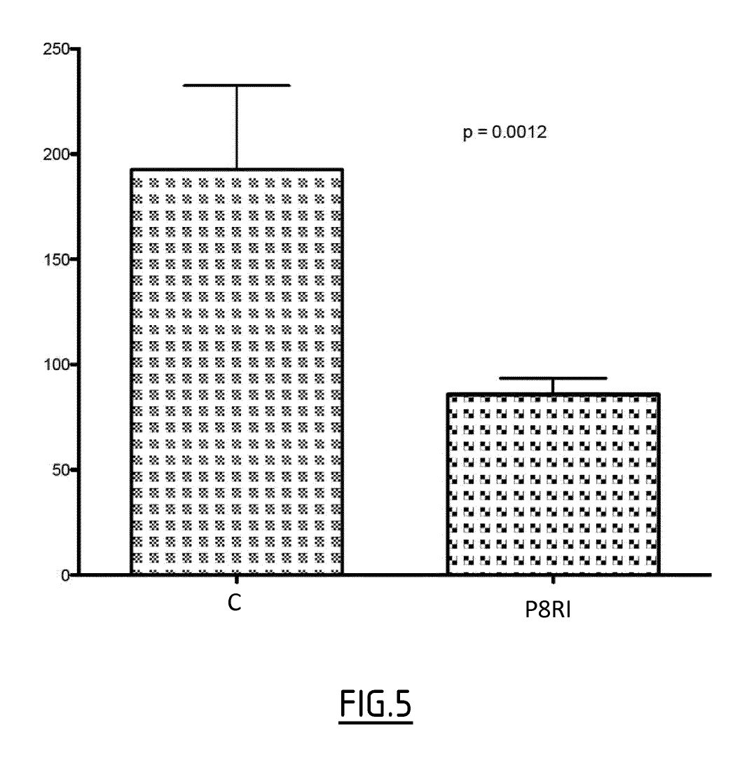

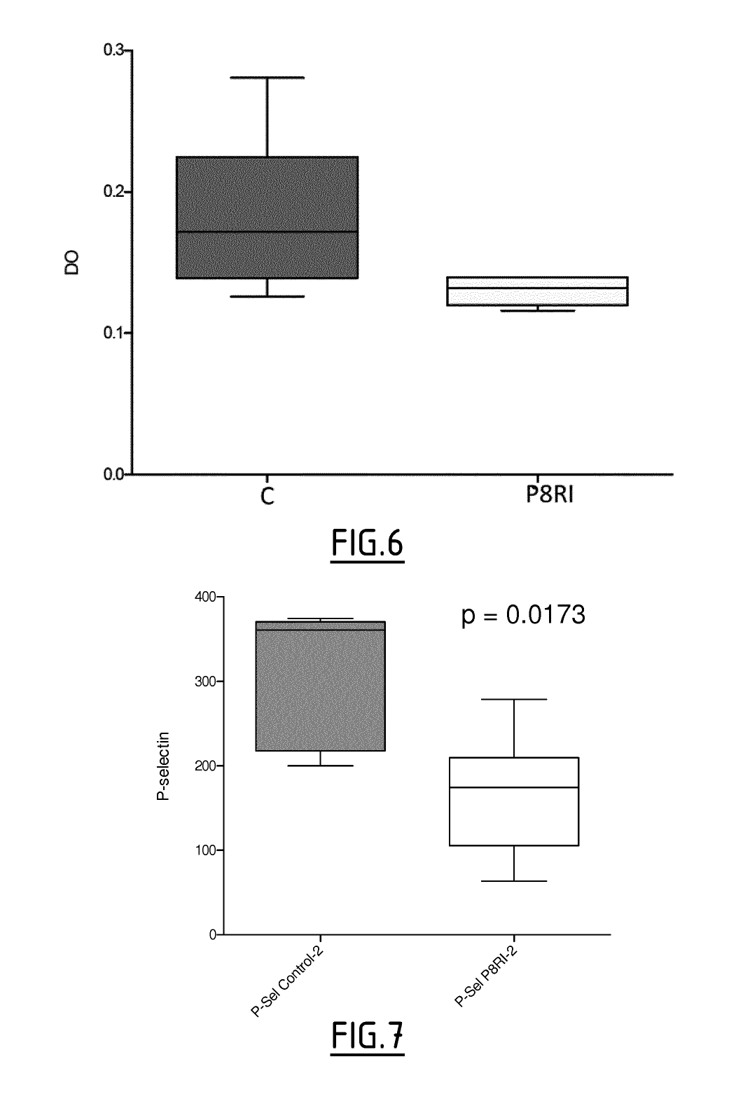

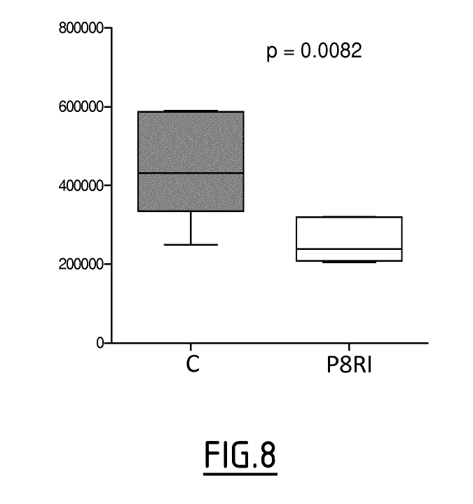

In these experimental conditions, P8RI perfusion significantly reduces the progressive increase in plasma and urinary free DNA in the rat plasma as compared to controls (FIGS. 3 and 4). P8RI perfusion is also capable to significantly decrease the hemoglobin content in the intestine lumen (129.4±47.39 versus 317.1±93.27 μg/mg prot, respectively, p<0.001, see FIG. 5), the accumulation and activation of neutrophils in the intestinal mucosa, as detected by the amount of MPO by direct ELISA (FIG. 6), the release of soluble P-selectin and MMP9 in the circulation (FIGS. 7 and 8) and to protect the epithelial villosity abrasion, epithelium desquamation and to partially preserve mucus film integrity. The administration of P8RI indeed preserved most of the mucin film by comparison to its destruction in control rats (data not shown).

1-14. (canceled)

15. A method for preventing and/or treating reperfusion injury in a subject in need thereof, wherein said method comprises the administration of a CD31^{shed} agonist to the subject.

16. The method according to claim 15, wherein said CD31^{shed} agonist is:
a) a peptide selected in the group consisting of:
(i) a peptide consisting of a fragment of 3 to 15 amino acids of the sequence defined by amino acids 579 to 601 of sequence SEQ ID NO: 1,
(ii) a peptide consisting of a fragment of 3 to 15 amino acids of a sequence corresponding to the amino acids 579 to 601 of sequence SEQ ID NO: 1 in a non-human mammalian CD31,
(iii) a peptide of 3 to 15 amino acids consisting of a sequence at least 70% identical to the sequence of peptide (i),
(iv) a peptide consisting of a retro-inverso sequence of peptide (i), (ii) or (iii), and
(v) the peptide (i), (ii), (iii) or (iv) comprising at least one or at least one further chemical modification,
or
b) a peptidomimetic of peptide a).

17. The method according to claim 16, wherein said peptide (v) comprises at least one amino acid in the D-enantiomer form.

18. The method according to claim 16, wherein said peptide is soluble in water.

19. The method according to claim 16, wherein said peptide is resistant to peptidase.

20. The method according to claim 16, wherein said peptide is selected in the group consisting of a peptide of sequence SEQ ID NO: 2, a peptide of sequence SEQ ID NO: 3, a peptide of sequence SEQ ID NO: 4, a peptide of sequence SEQ ID NO: 5, a peptide of sequence SEQ ID NO: 6 consisting of D-enantiomer amino acids, a peptide of sequence SEQ ID NO: 7 and a peptide of sequence SEQ ID NO: 8 consisting of D-enantiomer amino acids.

21. The method according to claim 16, wherein said peptide is a peptide of sequence SEQ ID NO: 5, or a peptide of sequence SEQ ID NO: 6 consisting of D-enantiomer amino acids.

22. The method according to claim 15, wherein said reperfusion is used for treating ischemia.

23. The method according to claim 15, wherein said reperfusion is used for treating ischemia and wherein said ischemia is selected in the group consisting of myocardial infarction, ischemic colitis, mesenteric ischemia, stroke, ischemia of the lower limbs, visceral ischemia resulting from acute hypovolemia, ischemia resulting from inflammatory conditions, ischemia inherent to an extracorporeal circulation blood excluding part of the arterial branches, ischemia inherent to a visceral and/or aortic surgery, cold ischemia, ischemia inherent to a warm reperfusion of a graft directed to organ transplantation, and their combinations.

24. The method according to claim 15, wherein said CD31^{shed} agonist is administered prior to and/or during reperfusion.

25. The method according to claim 1, wherein said CD31^{shed} agonist is administered intravenously.

26. The method according to claim 15, wherein said CD31^{shed} agonist is administered continuously during reperfusion.

27. The method according to claim 15, wherein a first bolus of said CD31^{shed} agonist is administered as a single dose, followed by a second bolus of said CD31^{shed} agonist administered continuously.

28. The method according to claim 15, wherein said CD31^{shed} agonist is administered at most for 48 hours.


SEQUENCE LISTING (74 sequences)

SEQ ID NO: 1 - protein, 738 aa, Homo sapiens
  SIGNAL (1)..(27)
  DOMAIN (28)..(601): Extracellular domain
  DOMAIN (34)..(121): First Ig-like domain
  DOMAIN (145)..(233): Second Ig-like domain
  DOMAIN (236)..(315): Third Ig-like domain
  DOMAIN (328)..(401): Fourth Ig-like domain
  DOMAIN (424)..(493): Fifth Ig-like domain
  DOMAIN (499)..(591): Sixth Ig-like domain
  DOMAIN (592)..(601): Juxta-membrane domain
  DOMAIN (602)..(620): Transmembrane domain
  DOMAIN (621)..(738): Cytoplasmic domain
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 2 - protein, 6 aa, Artificial Sequence
  muse or human-derived CD31 peptide
LAPWKK

SEQ ID NO: 3 - protein, 10 aa, Artificial Sequence
  mouse-derived CD31 peptide
VRVFLAPWKK

SEQ ID NO: 4 - protein, 10 aa, Artificial Sequence
  human-derived CD31 peptide
VRVILAPWKK

SEQ ID NO: 5 - protein, 8 aa, Artificial Sequence
  murine-derived CD31 peptide
RVFLAPWK

SEQ ID NO: 6 - protein, 8 aa, Artificial Sequence
  murine-derived CD31 peptide
KWPALFVR

SEQ ID NO: 7 - protein, 8 aa, Artificial Sequence
  human-derived CD31 peptide
RVILAPWK

SEQ ID NO: 8 - protein, 8 aa, Artificial Sequence
  human-derived CD31 sequence
KWPALIVR

SEQ ID NO: 9 - protein, 727 aa, Mus musculus
MLLALGLTLVLYASLQAEENSFTINSIHMESLPSWEVMNGQQLTLECLVDISTTSKSRSQHRVLFYKDDAMVYNVTSREHTESYVIPQARVFHSGKYKCTVMLNNKEKTTIEYEVKVHGVSKPKVTLDKKEVTEGGVVTVNCSLQEEKPPIFFKIEKLEVGTKFVKRRIDKTSNENFVLMEFPIEAQDHVLVFRCQAGILSGFKLQESEPIRSEYVTVQESFSTPKFEIKPPGMIIEGDQLHIRCIVQVTHLVQEFTEIIIQKDKAIVATSKQSSEAVYSVMAMVEYSGHYTCKVESNRISKASSIMVNITELFPKPKLEFSSSRLDQGELLDLSCSVSGTPVANFTIQKEETVLSQYQNFSKIAEESDSGEYSCTAGIGKVVKRSGLVPIQVCEMLSKPSIFHDAKSEIIKGHAIGISCQSENGTAPITYHLMKAKSDFQTLEVTSNDPATFTDKPTRDMEYQCRADNCHSHPAVFSEILRVRVIAPVDEVVISILSSNEVQSGSEMVLRCSVKEGTSPITFQFYKEKEDRPFHQAVVNDTQAFWHNKQASKKQEGQYYCTASNRASSMRTSPRSSTLAVRVFLAPWKKGLIAVVVIGVVIATLIVAAKCYFLRKAKAKQKPVEMSRPAAPLLNSNSEKISEPSVEANSHYGYDDVSGNDAVKPINQNKDPQNMDVEYTEVEVSSLEPHQALGTRATETVYSEIRKVDPNLMENRYSRTEGSLNGT

SEQ ID NO: 10 - protein, 739 aa, Bos taurus
MQLRWTQRGMMWLGALLTLLLCSSLKGQENSFTINSIHMQILPHSTVQNGENLTLQCLVDVSTTSRVKPLHQVLFYKDDVLLHNVSSRRNTESYLIPHVRVCDSGRYKCNVILNNKEKTTPEYEVWVKGVSDPRVTLDKKEVIEGGVVVVNCSVPEEKAPVHFTIEKFELNIRGAKKKREKTSQNQNFVTLEFTVEEQDRTIRFQCQAKIFSGSNVESSRPIQSDLVTVRESFSNPKFHIIPEGKVMEGDDLQVKCTVQVTHQAQSFPEIIIQKDREIVAHNSLSSEAVYSVMATTEHNGNYTCKVEASRISKVSSVVVNVTELFSKPKLESSATHLDQGEDLNLLCSIPGAPPANFTIQKGGMTVSQTQNFTKRVSEWDSGLYTCVAGVGRVFKRSNTVQITVCEMLSKPSIFHDSRSEVIKGQTIEVSCQSVNGTAPIFYQLSNTSKPVANQSVGSNKPAIFRVKPTKDVEYCCSADNCHSHSKMFSEVLRVKVIAPVDEAQLVVLKGEVEPGEPIVFYCSVNEGSFPITYKFYKEKESKPFYQDTINATQIMWHKTTASKEYEGQYYCTASNRANLSKHVIQSNTLTVRVYLPLEKGLIAVVVIGVIIVTLVLGAKCYFLKKAKAKQMPVEMSRPAVPLLNSNNEKTLSDAGTEADRHYGYNEDVGNHAMKPLNENKEPLTLDVEYTEVEVTSPEPHQGLGTKGTETETVYSEIRKADPDFVENRYSRTEGSLDGS

SEQ ID NO: 11 - protein, 740 aa, Sus scrofa
MRLRWTQGGNMWLGVLLTLQLCSSLEGQENSFTINSIHMEMLPGQEVHNGENLTLQCIVDVSTTSSVKPQHQVLFYKDDVLFHNVSSTKNTESYFISEARVYNSGRYKCTVILNNKEKTTAEYKVVVEGVSNPRVTLDKKEVIEGGVVKVTCSVPEEKPPVHFIIEKFELNVRDVKQRREKTANNQNSVTLEFTVEEQDRVILFSCQANVIFGTRVEISDSVRSDLVTVRESFSNPKFHISPKGVIIEGDQLLIKCTIQVTHQAQSFPEIIIQKDKEIVAHSRNGSEAVYSVMATVEHNSNYTCKVEASRISKVSSIMVNITELFSRPKLKSSATRLDQGESLRLWCSIPGAPPEANFTIQKGGMMMLQDQNLTKVASERDSGTYTCVAGIGKVVKRSNEVQIAVCEMLSKPSIFHDSGSEVIKGQTIEVSCQSINGTSPISYQLLKGSDLLASQNVSSNEPAVFKDNPTKDVEYQCIADNCHSHAGMPSKVLRVKVIAPVEEVKLSILLSEEVESGQAIVLQCSVKEGSGPITYKFYKEKENKPFHQVTLNDTQAIWHKPKASKDQEGQYYCLASNRATPSKNFLQSNILAVRVYLAPWKKGLIAVVVIAVIIAVLLLGARFYFLKKSKAKQMPVEMCRPAAPLLNSNNEKTLSDPNTEANRHYGYNEDVGNHAMKPLNENKEPLTLDVEYTEVEVTSPEPHRGLGTKGTETVYSEIRKADPDLVENRYSRTEGSLDGT

SEQ ID NO: 12 - protein, 23 aa, Artificial Sequence
  human-derived CD31 peptide
NHASSVPRSKILTVRVILAPWKK

SEQ ID NO: 13 - protein, 15 aa, Artificial Sequence
  peptide
SSTLAVRVFLAPWKK

SEQ ID NO: 14 - protein, 14 aa, Artificial Sequence
  peptide
STLAVRVFLAPWKK

SEQ ID NO: 15 - protein, 13 aa, Artificial Sequence
  peptide
TLAVRVFLAPWKK

SEQ ID NO: 16 - protein, 12 aa, Artificial Sequence
  peptide
LAVRVFLAPWKK

SEQ ID NO: 17 - protein, 11 aa, Artificial Sequence
  peptide
AVRVFLAPWKK

SEQ ID NO: 18 - protein, 9 aa, Artificial Sequence
  peptide
RVFLAPWKK

SEQ ID NO: 19 - protein, 8 aa, Artificial Sequence
  peptide
VFLAPWKK

SEQ ID NO: 20 - protein, 7 aa, Artificial Sequence
  peptide
FLAPWKK

SEQ ID NO: 21 - protein, 5 aa, Artificial Sequence
  peptide
APWKK

SEQ ID NO: 22 - protein, 4 aa, Artificial Sequence
  peptide
PWKK

SEQ ID NO: 23 - protein, 15 aa, Artificial Sequence
  peptide
SKILTVRVILAPWKK

SEQ ID NO: 24 - protein, 14 aa, Artificial Sequence
  peptide
KILTVRVILAPWKK

SEQ ID NO: 25 - protein, 13 aa, Artificial Sequence
  peptide
ILTVRVILAPWKK

SEQ ID NO: 26 - protein, 12 aa, Artificial Sequence
  peptide
LTVRVILAPWKK

SEQ ID NO: 27 - protein, 11 aa, Artificial Sequence
  peptide
TVRVILAPWKK

SEQ ID NO: 28 - protein, 9 aa, Artificial Sequence
  peptide
RVILAPWKK

SEQ ID NO: 29 - protein, 8 aa, Artificial Sequence
  peptide
VILAPWKK

SEQ ID NO: 30 - protein, 7 aa, Artificial Sequence
  peptide
ILAPWKK

SEQ ID NO: 31 - protein, 15 aa, Artificial Sequence
  peptide
SSMRTSPRSSTLAVR

SEQ ID NO: 32 - protein, 14 aa, Artificial Sequence
  peptide
SSMRTSPRSSTLAV

SEQ ID NO: 33 - protein, 13 aa, Artificial Sequence
  peptide
SSMRTSPRSSTLA

SEQ ID NO: 34 - protein, 12 aa, Artificial Sequence
  peptide
SSMRTSPRSSTL

SEQ ID NO: 35 - protein, 11 aa, Artificial Sequence
  peptide
SSMRTSPRSST

SEQ ID NO: 36 - protein, 10 aa, Artificial Sequence
  peptide
SSMRTSPRSS

SEQ ID NO: 37 - protein, 9 aa, Artificial Sequence
  peptide
SSMRTSPRS

SEQ ID NO: 38 - protein, 8 aa, Artificial Sequence
  peptide
SSMRTSPR

SEQ ID NO: 39 - protein, 7 aa, Artificial Sequence
  peptide
SSMRTSP

SEQ ID NO: 40 - protein, 6 aa, Artificial Sequence
  peptide
SSMRTS

SEQ ID NO: 41 - protein, 5 aa, Artificial Sequence
  peptide
SSMRT

SEQ ID NO: 42 - protein, 4 aa, Artificial Sequence
  peptide
SSMR

SEQ ID NO: 43 - protein, 15 aa, Artificial Sequence
  peptide
NHASSVPRSKILTVR

SEQ ID NO: 44 - protein, 14 aa, Artificial Sequence
  peptide
NHASSVPRSKILTV

SEQ ID NO: 45 - protein, 13 aa, Artificial Sequence
  peptide
NHASSVPRSKILT

SEQ ID NO: 46 - protein, 12 aa, Artificial Sequence
  peptide
NHASSVPRSKIL

SEQ ID NO: 47 - protein, 11 aa, Artificial Sequence
  peptide
NHASSVPRSKI

SEQ ID NO: 48 - protein, 10 aa, Artificial Sequence
  peptide
NHASSVPRSK

SEQ ID NO: 49 - protein, 9 aa, Artificial Sequence
  peptide
NHASSVPRS

SEQ ID NO: 50 - protein, 8 aa, Artificial Sequence
  peptide
NHASSVPR

SEQ ID NO: 51 - protein, 7 aa, Artificial Sequence
  peptide
NHASSVP

SEQ ID NO: 52 - protein, 6 aa, Artificial Sequence
  peptide
NHASSV

SEQ ID NO: 53 - protein, 5 aa, Artificial Sequence
  peptide
NHASS

SEQ ID NO: 54 - protein, 4 aa, Artificial Sequence
  peptide
NHAS

SEQ ID NO: 55 - protein, 15 aa, Artificial Sequence
  peptide
TSPRSSTLAVRVFLA

SEQ ID NO: 56 - protein, 13 aa, Artificial Sequence
  peptide
SPRSSTLAVRVFL

SEQ ID NO: 57 - protein, 11 aa, Artificial Sequence
  peptide
PRSSTLAVRVF

SEQ ID NO: 58 - protein, 9 aa, Artificial Sequence
  peptide
RSSTLAVRV

SEQ ID NO: 59 - protein, 7 aa, Artificial Sequence
  peptide
SSTLAVR

SEQ ID NO: 60 - protein, 5 aa, Artificial Sequence
  peptide
STLAV

SEQ ID NO: 61 - protein, 15 aa, Artificial Sequence
  peptide
SVPRSKILTVRVILA

SEQ ID NO: 62 - protein, 13 aa, Artificial Sequence
  peptide
VPRSKILTVRVIL

SEQ ID NO: 63 - protein, 11 aa, Artificial Sequence
  peptide
PRSKILTVRVI

SEQ ID NO: 64 - protein, 9 aa, Artificial Sequence
  peptide
RSKILTVRV

SEQ ID NO: 65 - protein, 7 aa, Artificial Sequence
  peptide
SKILTVR

SEQ ID NO: 66 - protein, 5 aa, Artificial Sequence
  peptide
KILTV

SEQ ID NO: 67 - protein, 4 aa, Artificial Sequence
  peptide
RVFL

SEQ ID NO: 68 - protein, 5 aa, Artificial Sequence
  peptide
RVFLA

SEQ ID NO: 69 - protein, 6 aa, Artificial Sequence
  peptide
RVFLAP

SEQ ID NO: 70 - protein, 7 aa, Artificial Sequence
  peptide
RVFLAPW

SEQ ID NO: 71 - protein, 4 aa, Artificial Sequence
  peptide
RVIL

SEQ ID NO: 72 - protein, 5 aa, Artificial Sequence
  peptide
RVILA

SEQ ID NO: 73 - protein, 6 aa, Artificial Sequence
  peptide
RVILAP

SEQ ID NO: 74 - protein, 7 aa, Artificial Sequence
  peptide
RVILAPW